SEQUENCE LISTING (1 sequence)

SEQ ID NO: 1 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2022/0251137 A1
(43) Pub. Date: Aug. 11, 2022

(54) Temperature-controlled purification of granulocyte-colony stimulating factor

(71) Applicants: Jennifer Renee Hopp, San Diego, CA (US); Tanvex BioPharma USA, Inc., San Diego, CA (US)

(72) Inventor: Jennifer Renee Hopp, San Diego, CA (US)

(21) Appl. No.: 17/625,655

(22) PCT Filed: Jul. 7, 2020
(86) PCT No.: PCT/US2020/041060

§ 371 Date: Jan. 7, 2022

Related U.S. Application Data
(60) Provisional application No. 62/871,905, filed on Jul. 9, 2019.

(51) Int. Cl. C07K 1/18; C07K 14/535; C07K 1/36; B01D 15/36; B01D 15/16; G01N 30/30; G01N 30/96

(52) U.S. Cl. CPC C07K 1/18; C07K 14/535; C07K 1/36; A61K 38/00; B01D 15/161; G01N 30/30; G01N 30/96; B01D 15/363

(57) ABSTRACT

Provided herein, inter alia, are compositions, systems, and methods for the purification of granulocyte colony-stimulating factor (GCSF). Also provided are GCSF obtained by the disclosed methods and systems, pharmaceutical compositions containing the same, as well as methods for the treatment and/or prevention of a disease or health condition in an individual in need thereof.

### CROSS-REFERENCE TO RELATED APPLICATIONS

This application claims the benefit of priority to U.S. Provisional Patent Application Ser. No. 62/871,905, filed on Jul. 9, 2019. The disclosure of the above-referenced application is herein expressly incorporated by reference it its entirety, including any drawings.

### FIELD

The present disclosure generally relates to a temperature-controlled anion exchange (AEX) chromatography process for the purification of granulocyte colony stimulating factor (GCSF).

### BACKGROUND

To ensure the safety of biopharmaceuticals, regulatory agencies impose stringent purification standards and relevant quality attributes (e.g., identity, purity, and biological activity) for recombinant proteins intended for human administration. Common standards require that protein-based therapeutic products are substantially free from impurities, including product related contaminants, such as aggregates, fragments, and inactive variants of the target recombinant protein, and process related contaminants, such as leached chromatography resin ligands, culture media components, host cell proteins and nucleic acids, viral contaminants, and endotoxins.

Ion-exchange chromatography (IEC) is one of the most commonly used techniques for the separation and purification of proteins, polypeptides, nucleic acids, polynucleotides, and other biomolecules. Some key reasons for using IEC are its broad relevance, high capacity, and the potentially high efficiency of the system. In the case of granulocyte-colony stimulating factor (GCSF), the isolation and purification of GCSF has long been studied in an effort to develop efficient, scalable, and industrially applicable purification processes, e.g. can be implemented at a commercial production scale. Application of IEC to the purification and preparation of GCSF has been an important step forward for this field. Several chromatographic parameters affecting the purification yield of GCSF, such as the buffering agent concentration, pH value, concentration and ratio of thiol pairs in the mobile phase, as well as the flow rate of the mobile phase, have been investigated in detail and indicated that these parameters are of great importance. However, low yields and/or inconsistent GCSF yields have been observed in many existing IEC operations, including anion exchange chromatography (AEX). These low and/or inconsistent yields represent some of the most challenging obstacles to designing and developing scaled-up GCSF manufacturing processes.

Thus, there is still an unmet need for improved production methods that are cost-effective, stable for high recovery of GCSF, and amenable for large-scale production.

### SUMMARY

Provided herein, inter alia, are compositions, methods, and systems for the purification and/or preparation of granulocyte colony-stimulating factor (GCSF) protein. Certain aspects and embodiments of the disclosure concern new approaches to optimize the purification of GCSF protein using an AEX procedure which is conducted under cold temperature conditions in order to obtain high and consistent GCSF yield. In particular, the inventor has demonstrated that using a jacketed chromatography column wherein at least a portion of the jacket is set at a temperature of about 7° C. to about 13° C. results in surprising improvement in yield of GCSF product. Also provided are GCSF obtained by such methods, systems, pharmaceutical compositions containing the same, as well as methods for the treatment and/or prevention of a disease or health condition in a subject in need thereof.

In one aspect, provided herein are various methods for purifying granulocyte colony-stimulating factor (GCSF), including (a) loading a GCSF-containing sample onto a chromatography vessel including an anion exchange chromatography (AEX) material capable of binding the GCSF in the sample, wherein the chromatography vessel is placed in a temperature-controlled enclosure and at least a portion of the enclosure of the chromatography vessel is set at a temperature of about 7° C. to about 13° C.; and (b) eluting the GCSF from the AEX material with an elution buffer to obtain the purified GCSF.

Non-limiting exemplary embodiments of the disclosed methods include one or more of the following features. In some embodiments, the GCSF-containing sample is loaded onto the chromatography vessel using a fluidic channel placed in a temperature-controlled enclosure, wherein at least a portion of the enclosure of the fluidic channel is set at a temperature about 7° C. to about 13° C. In some embodiments, the set temperatures of the enclosure of the vessel and the enclosure of the fluidic channel are the same. In some embodiments, the set temperatures of the enclosure of the chromatography vessel and the enclosure of the fluidic channel jacket are different. In some embodiments, at least one of the enclosure of the chromatography vessel and the enclosure of the fluidic channel is set at a temperature of about 10° C. In some embodiments, the chromatography vessel is selected from the group consisting of a column, a tank, a packed bed, a fluidized bed, a cartridge, an encapsulated membrane, a reservoir, a chamber, a container, and a mixing vessel. In some embodiments, the fluidic channel is a tube, a pipe, a bag, a container, a storage tank, a mixing vessel, or other fluid conduction means.

In some embodiments, the method further includes, prior to loading of the GCSF sample, equilibrating the AEX material with an equilibration buffer including from about 30 mM to about 50 mM Tris, and at pH of about 7.0 to about 8.0. In some embodiments, the equilibration buffer includes about 40 mM Tris and at pH of about 7.6. In some embodiments, the method further includes, prior to elution of the GCSF, washing the AEX material with a washing buffer to remove unbound or weakly bound contaminants. In some embodiments, the wash buffer and the equilibration buffer have the same buffer composition. In some embodiments, the GCSF-containing sample includes a loading buffer. In some embodiments, the loading buffer has a pH of about 7.4 to about 8.0. In some embodiments, the loading of the GCSF sample onto the chromatography vessel is carried out at a conductivity ranging between about 1.5 to about 3.0 mS/cm.

In some embodiments, the elution buffer includes about 30 mM-60 mM Tris, about 30 mM-80 mM sodium chloride, and a pH of about 7.4 to about 8.0. In some embodiments, the elution buffer includes about 40 mM Tris, about 50 mM sodium chloride, and pH of about 7.7. In some embodiments, the elution of the GCSF from the AEX material is carried out at a conductivity ranging between about 7.4 to about 8.2 mS/cm. In some embodiments, the AEX material includes diethylaminoethyl (DEAE) ion-exchange chromatography. In some embodiments, the AEX material includes DEAE Sepharose® resin. In some embodiments, the DEAE Sepharose® resin includes DEAE Sepharose® Fast Flow resin.

In some embodiments, the method further includes one or more phases of stripping and/or sanitation of the AEX material. In some embodiments, the DEAE chromatography is operated at a linear flow rate for all phases. In some embodiments, the linear flow rate is about 150 cm/hr. In some embodiments, at least one of the stripping and sanitization phases is performed at 50 cm/hr. In some embodiments, the GCSF is a recombinant human GCSF (hGCSF) or a variant thereof. In some embodiments, the sample includes GCSF obtained from a recombinant eukaryotic cell or a recombinant prokaryotic cell.

In some embodiments, the methods disclosed herein further include at least one additional purification process. In some embodiments, the at least one additional purification process is selected from the group consisting of affinity chromatography, cation exchange chromatography (CEX), hydroxyapatite chromatography, size exclusion chromatography (SEC), hydrophobic interaction chromatography (HIC), metal affinity chromatography, mixed mode chromatography (MMC), centrifugation, diafiltration, and ultrafiltration. In some embodiments, the at least one additional purification process is performed prior to the AEX chromatography process. In some embodiments, the at least one additional purification process is performed after the AEX chromatography process.

In another aspect, provided herein are various systems for manufacturing GCSF that include a chromatography vessel including an anion exchange chromatography (AEX) material capable of binding GCSF, wherein the chromatography vessel is encased in a temperature-controlled enclosure and at least a portion of the enclosure of the chromatography vessel is set a temperature of about 7° C. to about 13° C.

Non-limiting exemplary embodiments of the disclosed systems include one or more of the following features. In some embodiments, the system further includes a fluidic channel placed in a temperature-controlled enclosure, wherein at least a portion of enclosure of the fluidic channel is set at a temperature 7° C. to about 13° C. In some embodiments, the set temperatures of the enclosure of the vessel and the enclosure of the fluidic channel are the same. In some embodiments, the set temperatures of the enclosure of the chromatography vessel and the enclosure of the fluidic channel jacket are different. In some embodiments, at least one of the enclosure of the chromatography vessel and the enclosure of the fluidic channel is set at a temperature of about 10° C. In some embodiments, the chromatography vessel is selected from the group consisting of a column, a tank, a packed bed, a fluidized bed, a cartridge, an encapsulated membrane, a reservoir, a chamber, a container, and a mixing vessel. In some embodiments, the fluidic channel is a tube, a pipe, a bag, a container, a storage tank, a mixing vessel, or other fluid conduction means.

In some embodiments, prior to loading of the GCSF sample, the AEX material is equilibrated with an equilibration buffer including from about 30 mM to about 50 mM Tris, and at pH of about 7.0 to about 8.0. In some embodiments, the equilibration buffer includes about 40 mM Tris and at pH of about 7.6. In some embodiments, prior to elution of the GCSF, the AEX material is washed with a wash buffer to remove unbound or weakly bound contaminants. In some embodiments, the wash buffer and the equilibration buffer have the same buffer composition. In some embodiments, the GCSF-containing sample includes a loading buffer. In some embodiments, the loading buffer has a pH of about 7.4 to about 8.0. In some embodiments, the loading of the GCSF sample onto the chromatography vessel is carried out at a conductivity ranging between about 1.5 to about 3.0 mS/cm.

In some embodiments of the disclosed systems, the elution buffer includes about 30 mM-60 mM Tris, about 30 mM-80 mM sodium chloride, and a pH of about 7.4 to about 8.0. In some embodiments, the elution buffer includes about 40 mM Tris, about 50 mM sodium chloride, and pH of about 7.7. In some embodiments, the elution of the GCSF from the AEX material is carried out at a conductivity ranging between about 7.4 to about 8.2 mS/cm. In some embodiments, the AEX material includes diethylaminoethyl (DEAE) ion-exchange chromatography. In some embodiments, the AEX material includes DEAE Sepharose® resin. In some embodiments, the DEAE Sepharose® resin includes DEAE Sepharose® Fast Flow resin.

In some embodiments, the systems further includes one or more phases of stripping and/or sanitation of the AEX material. In some embodiments, the DEAE chromatography is operated at a linear flow rate for all phases. In some embodiments, the linear flow rate is about 150 cm/hr. In some embodiments, at least one of the stripping and sanitization phases is performed at 50 cm/hr. In some embodiments, the GCSF is a recombinant human GCSF (hGCSF) or a variant thereof. In some embodiments, the sample includes GCSF obtained from a recombinant eukaryotic cell or a recombinant prokaryotic cell.

In some embodiments, the systems disclosed herein further include at least one additional purification process. In some embodiments, the at least one additional purification process is selected from the group consisting of affinity chromatography, cation exchange chromatography (CEX), hydroxyapatite chromatography, size exclusion chromatography (SEC), hydrophobic interaction chromatography (HIC), metal affinity chromatography, mixed mode chromatography (MMC), centrifugation, diafiltration, and ultrafiltration. In some embodiments, the at least one additional purification process is performed prior to the AEX chromatography process. In some embodiments, the at least one additional purification process is performed after the AEX chromatography process.

In another aspect, some embodiments of the disclosure relate to a granulocyte colony-stimulating factor (GCSF) purified by a method disclosed herein, or a system disclosed herein. In a related aspect, some embodiments of the disclosure relate to a pharmaceutical composition including a GCSF as disclosed herein. In some embodiments, the pharmaceutical compositions is an aqueous composition, a lyophilisate, or a powder.

In yet another aspect, provided herein are various methods for treating or preventing a disease or health condition in a subject in need thereof, the methods including administering to the subject a GCSF as disclosed herein and/or a pharmaceutical composition as disclosed herein. In some embodiments, the disease or health condition is neutropenia.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### BRIEF DESCRIPTION OF THE DRAWINGS

FIG. 1 graphically illustrates the distribution of GCSF product quality across the DEAE elution peak in an exemplary experiment in accordance with some embodiments of the methods disclosed herein. In this experiment, collection of fractions of 0.5 CV in volume from the DEAE resin was initiated at 0.5 OD at 280 nm and stopped when the OD dropped to 0.5. These fractions were subsequently analyzed by reversed phase (RP) and for host cell protein content.

### DETAILED DESCRIPTION OF THE DISCLOSURE

Provided herein, inter alia, are compositions, methods, and systems for the purification and/or preparation of granulocyte colony-stimulating factor (GCSF) protein. Certain aspects and embodiments of the disclosure concern new approaches to optimize the purification of GCSF protein using an AEX procedure which is conducted under cold temperature conditions in order to obtain high and consistent GCSF yield.

In ion-exchange chromatography (IEC), temperature of the chromatography column has been reported as a factor influencing the selectivity of ion exchange reactions and the efficiency of the column, and thus influences the quality of the separation of ions. For example, in classical IEC, temperature of the column has been evidenced as a factor influencing the selectivity towards inorganic ions. As described in greater detail below, incorporation of a bind-and-elute AEX operation including a temperature-controlled chromatography vessel, such as a chromatography vessel encased in a temperature-controlled enclosure, with the temperature of the enclosure set a specific temperature ranging from about 7° C. to about 13° C. has allowed high and/or consistent yields of GCSF product, which therefore can suitably be used for the manufacture of GCSF. As described in the Examples below, a number of robustness studies have been performed to evaluate and optimize various parameters for the preparation of recombinant hGCSF in a temperature-controlled DEAE chromatography process. These parameters include (1) load density, (2) load pH, (3) load conductivity, (4) equilibration pH, (5) equilibration conductivity, (6) elution pH, (7) elution conductivity, (8) load jacket set point temperature, and (9) column jacket set point temperature. In these experiments, DEAE functions as a capture chromatography column with potential capability of reducing oxidative species, DNA, and E. coli host cell protein (ECP). Experimental data presented herein indicate that certain parameters have an significant influence on GCSF product quality and/or yield. For example, the jacket temperature set-points for both the chromatography vessel and the load have been identified as key process parameters (KPP) for having significant influence on the yield and quality of the final GCSF product (see, e.g., Example 3 and Table 13). In particular, the jacket temperature of the chromatography vessel was found to have an influence on the product-related impurities as determined by a C3 Reverse Phase HPLC assay, and also have an influence on the charge heterogeneity of GCSF final product as determined by a CEX HPLC assay. In addition, the load jacket temperature was found to have an influence on the host cell protein content in the final GCSF product.

As further explained below, the methods of preparing GCSF disclosed herein can suitably provide an effective production process at an industrial scale for recombinant GCSF of high purity, e.g., up to pharmaceutical grade, considering quality, economy, and regulatory needs. Also provided herein, in some embodiments of the disclosure, are GCSF obtained by such methods, pharmaceutical compositions containing the same, as well as methods for the treatment and/or prevention of a disease or health condition in a subject in need thereof.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof “A and/or B” is used herein to include all of the following alternatives: “A”, “B”, “A or B”, and “A and B”.

Certain values and ranges are presented herein with numerical values being preceded by the term “about.” The term “about” is used herein to provide literal support for the exact number that it precedes, as well as a number that is near to or approximately the number that the term precedes. Generally, the term “about” has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values

It is understood that aspects and embodiments of the disclosure described herein include “comprising,” “consisting,” and “consisting essentially of” aspects and embodiments.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Headings, e.g., (a), (b), (i) etc., are presented merely for ease of reading the specification and claims. The use of headings in the specification or claims does not require the steps or elements be performed in alphabetical or numerical order or the order in which they are presented.

### Granulocyte Colony-Stimulating Factor (GCSF)

Granulocyte colony-stimulating factor (GCSF) is a multifunctional cytokine which is widely used for treating neutropenia in humans. GCSF is a hematopoietic lineage-specific cytokine mainly produced by fibroblasts and endothelial cells from bone marrow stroma and by immunocompetent cells (such as, e.g., monocytes, macrophages). The receptor for GCSF (GCSFR) is part of the cytokine and hematopoietin receptor superfamily and GCSFR mutations cause severe congenital neutropenia. The main action of GCSF/GCSFR linkage is stimulation of the differentiation, proliferation, mobilization, survival, and chemotaxis of neutrophils in the bone marrow and control their release to the bloodstream. In addition, many other GCSF effects have been reported, including (i) growth and migration of endothelial cells, (ii) decrease of norepinephrine reuptake, (iii) increase in osteoclastic activity, and (iv) decrease in osteoblast activity.

The human GCSF gene located on human chromosome 17 encodes two protein products due to alternative splicing: isoform A composed of 177 amino acids and isoform B of 174 amino acids. Isoform A contains an additional three residues (Val-Ser-Gln) inserted after Leu35 in isoform B. Isoform B has greater biological activity and stability than those of isoform A. Thus, human (h)GCSF, isoform B, has been mainly targeted for cloning and expression. The hGCSF is an 18.8-kDa glycoprotein that has 5 cysteine residues as well as 2 intra-molecular disulfide bonds that are indispensable to its biological activity. Recombinant (r)hGCSF produced by Escherichia coli (E. coli) has similar biological activity to that of the native human protein, but differs in that it contains an N-terminal methionine residue and is not glycosylated. The expression of rhGCSF in E. coli often results in the formation of insoluble aggregates, which are called inclusion bodies (IBs). In existing purification processes, these insoluble aggregates are solubilized in a buffer containing denaturing agents, generally followed by a renaturation step to transform the denatured forms into biologically active forms with apposite three-dimensional structure.

The therapeutic indications of GCSF have been widely reported and include non-neutropenic patient infections, reproductive medicine, neurological disturbances, regeneration therapy after acute myocardial infarction and of skeletal muscle, and hepatitis C therapy. Currently, GCSF is used clinically to facilitate hematopoietic recovery after bone marrow transplantation or cancer chemotherapy. In oncology, GCSF is utilized especially for the primary prophylaxis of chemotherapy-induced neutropenia, but it can be used for hematopoietic stem cell transplantation, wherein it can produce monocytic differentiation of some myeloid leukemias.

### Methods for Purifying GCSF

Earlier reports indicate that the levels of rhGCSF expressed in recombinant prokaryotic cells, such as E. coli were at low to moderate levels (10-35%), and the yield of the final product was very poor and far from acceptable. These low yields can be due to a number of factors, including unproductive downstream process technologies such as isolation of inclusion bodies with low purity, recovery, misfolding, aggregate formation, and un-optimized conditions of protein refolding operations, and chromatographic processes. Thus, there is still a need for improved production methods that are scalable, cost-effective, and stable for high recovery of GCSF.

Certain aspects and embodiments of the disclosure concern new approaches to optimize the purification of GCSF protein using an AEX procedure conducted under cold temperature conditions, in order to obtain high and consistent GCSF yield. Some embodiments of the disclosure relate to a method for the purification GCSF using a bind-and-elute operation and chromatography vessel placed in a temperature-controlled enclosure suitably equipped for temperature-controlled operations. In some embodiments, the disclosed method includes (i) loading a GCSF-containing sample onto a chromatography vessel containing an AEX material capable of binding the GCSF in the sample, wherein the chromatography vessel is encased in a temperature-controlled enclosure and at least a portion of the enclosure of the chromatography vessel is set at a temperature of about 7° C. to about 13° C.; and (ii) eluting the GCSF from the AEX material with an elution buffer. In some embodiments, the GCSF present in the sample binds to the AEX material in a reversible manner. Those skilled in the art will understand that binding a molecule of interest (e.g., GCSF) to a chromatography material refers to exposing the molecule to chromatography material under appropriate conditions (e.g., pH and/or conductivity) such that the molecule is reversibly immobilized in or on the chromatography material by virtue of ligand-protein interactions. Non-limiting examples include ionic interactions between the molecule and a charged group or charged groups of the ion exchange material.

The term purification in this application refers to a procedure to increase the degree of purity of the GCSF protein in a GCSF-containing sample by which the concentration of one or more undesired contaminant compounds (e.g., any foreign or undesirable impurities) is reduced relative to the concentration of GCSF protein. The term GCSF-containing sample refers to a mixture which comprises the GCSF protein in the presence of one or more contaminant compounds (impurities). As discussed in greater detail below, the GCSF protein to be purified using the methods described herein may be generally produced using recombinant techniques in eukaryotic organisms (e.g., yeast and mammalian cell lines) or in prokaryotic organism such as bacteria (e.g., E. coli). Contaminant compounds present in a GCSF-containing sample can be product related contaminants, such as aggregates, fragments, and inactive variants of the target protein (e.g., GCSF). Contaminant compounds can also be process related contaminants, such as leached chromatography resins, culture media components, cell debris, host cell proteins, salts, lipids carbohydrates, nucleic acids, viral contaminants, and endotoxins.

Depending on the starting material for the process; the undesired contaminant compounds can be completely or partially removed from the GCSF product. As described in greater detail below, incorporation of a bind-and-elute AEX operation including a temperature-controlled chromatography vessel, such as a jacketed chromatography vessel, with the temperature of the vessel jacket set a specific temperature ranging from about 7° C. to about 13° C. has allowed high and/or consistent yields of GCSF product, which can suitably be used for the industrial manufacture of GCSF.

### Anion Exchange Chromatography (AEX)

Non-limiting exemplary embodiments of the disclosed methods include incorporation of a bind-and-elute AEX operation conducted under cold temperature conditions. AEX is a separation technique commonly used widely to isolate and/or purify biomolecules such as proteins, amino acids, sugars/carbohydrates and other acidic substances with a negative charge at higher pH levels. AEX separate substances based on their charges using a solid-phase ion exchange material (e.g., matrix, media, or resin) containing positively charged groups. The charge may be provided by attaching one or more anion-exchanger groups, such as diethyl aminoethyl (DEAE), trimethyl hydroxypropyl (QA), quaternary aminoethyl (QAE), quaternary aminomethyl (Q), diethyl-(2-hydroxypropyl)-aminoethyl, triethyl aminomethyl (TEAE), triethylaminopropyl (TEAP), polyethyleneimine (PEI), to the solid phase, e.g., by covalent linking. Alternatively, or in addition, the charge may be an inherent property of the solid phase (e.g., as is the case for silica, which has an overall positive charge). In solution, the AEX material is coated with positively charged counter-ions (cations). AEX material binds to negatively charged molecules, displacing the counter-ions. The tightness of the binding between the substances and the resin is based on the strength of the negative charge of the substances.

Generally, the AEX material for use in the methods of the disclosure can be any AEX material containing functional groups (e.g., anion exchangers) suitable for AEX chromatography of proteins. Non-limiting examples of functional AEX groups suitable for the disclosed methods include diethyl aminoethyl (DEAE), trimethyl hydroxypropyl (QA), quaternary aminoethyl (QAE), quaternary aminomethyl (Q), diethyl-(2-hydroxypropyl)-aminoethyl, triethyl aminomethyl (TEAE), triethylaminopropyl (TEAP), polyethyleneimine (PEI). Examples of suitable commercially available products include, but are not limited to, DEAE-Sepharose FF, DEAE-Sepharose CL-4B, Q-Sepharose FF, Q-Sepharose CL-4B, Q-Sepharose HP, Q-Sepharose XL, Q-Sepharose Big Beads, QAE-Sephadex, DEAE-Sephadex, Capto DEAE, Capto Q, Capto Q ImpRes, Source 15Q, Source 30Q, DEAE Sephacel, Macro-Prep High Q, Macro-Prep DEAE, Nuvia Q, TOYOPEARL DEAE-650, TOYOPEARL SuperQ-650, TOYOPEARL QAE-550, Fractogel EMD DEAE, Fractogel EMD TMAE, Biosepra Q Ceramic HyperD, and Biosepra DEAE Ceramic HyperD. Other examples of suitable AEX materials known in the art include, but are not limited to Poros HQ 50, Poros PI 50, Poros D, Sartobind Q, and Mustang Q.

The selection of the AEX material for use in the disclosed methods can be made on the basis of the desired separation performance, process times, cleaning robustness, reproducibility, binding capacity, lot-to-lot consistency, and overall economy, etc. The skilled person will appreciate that besides the functional AEX group, the nature of the backbone of the AEX resin as well as the size of the beads also needs to be considered. In some embodiments, a matrix based on methacrylate derivatives such as, e.g., Macro-Prep® and Toyopearl® can be used. Such a matrix has been reported to possess particularly good resolution and reproducibility. In some embodiments, cross-linked agarose matrices such as, for example Sepharose©, can be used. Since the AEX chromatography is used in a bind-and-elute operation, selective elution conditions can be used, which can be either provided by increasing the salt concentration by steps or gradients, or by decreasing the pH in the elution buffer by steps or gradients.

In some embodiments, the AEX material includes weak anion exchangers such as, for example DEAE functional groups. DEAE is a classical weak anion exchange group, which in the experiments described below showed good resolution and fast equilibration profiles. In some embodiments, an AEX chromatography with DEAE Sepharose FF is performed to allow high flow rates and good product recovery.

In some embodiments, the AEX material includes DEAE ion-exchange chromatography. In some embodiments, the anion exchange material includes DEAE Sepharose® resin. In some embodiments, the DEAE Sepharose® resin includes DEAE Sepharose® Fast Flow resin.

The AEX material suitable for use in the methods disclosed herein can generally be any form of solid phase which can separate an analyte of interest (e.g., a GCSF polypeptide) from other molecules present in a mixture. Generally, the analyte of interest can be separated from other molecules as a result of differences in rates at which the individual molecules of the mixture migrate through a stationary solid phase under the influence of a moving phase. Alternatively, the analyte of interest can be separated from other molecules in a bind-and-elute operation. In some embodiments, the AEX operation is performed in a bind-and-elute mode, where binding of GCSF (i.e., the analyte of interest) to an AEX material involves contacting GCSF to the AEX material under appropriate conditions (e.g., pH, temperature, and/or conductivity) such that GCSF is reversibly immobilized in or on the chromatography material by virtue of ionic interactions between GCSF and a charged group or charged groups of the AEX material.

Generally, unbound impurities can then be collected in the flow-through and/or in subsequent buffer washes. GCSF molecules that bind to the positively charged AEX material are retained and can subsequently be eluted by at least one of two commonly used techniques. In one technique, in some embodiments, GCSF molecules that are bound to the AEX material can be eluted when the salt concentration in the elution buffer is gradually increased. In this case, the negative ions in the salt solution (e.g., Cl⁻) compete with protein in binding to the AEX material. In a second technique, in other embodiments, GCSF molecules bound to the AEX material can be eluted when the pH of the solution is gradually decreased which results in a more positive charge on the GCSF, releasing it from the AEX material. Both of these techniques can displace the negatively charged GCSF which is then eluted into test tubes fractions with the elution buffer.

Non-limiting examples of AEX materials suitable for the methods of the disclosure include AEX resins, AEX matrix, AEX media, and AEX membranes. In some embodiments, the AEX material used in the disclosed methods can be a resin. In some embodiments, the AEX material used in the disclosed methods can comprise one or more of a primary amine, a secondary amine, a tertiary amine, a quaternary ammonium ion functional group, a polyamine functional group, and a diethylaminoethyl functional group. In some embodiments, the AEX material used in the disclosed methods is packed in a chromatography vessel (e.g., column). In some embodiments, the AEX material is an AEX membrane.

Those skilled in the art will appreciate that the volume of the AEX material, the length, and the diameter of the chromatography vessel (e.g., column) to be used, as well as the dynamic capacity and flow-rate will depend on several parameters such as the volume of fluid to be treated, and concentration of GCSF protein in the samples to be subjected to the purification methods of the disclosure. Determination of these parameters is well within the average skills of those skilled in the art.

### Temperature-Controlled Vessel

As described above, some embodiments of the disclosure concern a method for the purification GCSF using an AEX chromatography system suitably equipped for temperature-controlled operations. In the disclosed methods, the chromatography vessel can generally be any suitable chromatographic apparatus that enables housing of the AEX material (e.g., resin, media, or matrix). Non-limiting examples of chromatography vessel suitable for use in the disclosed methods include chromatography columns, tanks, packed beds, fluidized beds, reservoirs, chambers, containers, and mixing vessels. Additional chromatography vessels suitable for use include, but are not limiting to, chromatographic cartridges such as, for example, encapsulated membranes. Generally, the chromatography vessel of the disclosure can have a cylindrical shape. In some embodiments of the disclosed methods, the chromatography vessel may have other shapes suitable for housing of the AEX material such as, for example, cylindrical shapes, ellipsoidal shapes, conical shapes, other rounded shapes, shapes with less-rounded wall segments, shapes with more straight wall segments, cuboidal shapes, other flatten shapes, or combinations thereof. In some embodiments, the chromatography vessel of the disclosure can be a cylindrical chromatography column or tank.

Generally, there are no particular limitations to the volume of the chromatography vessel. In some embodiments, the chromatography vessel can have a volume of greater than about 1 mL, about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 40 mL, about 50 mL, about 75 mL, about 100 mL, about 200 mL, about 300 mL, about 400 mL, about 500 mL, about 600 mL, about 700 mL, about 800 mL, about 900 mL, about 1 L, 2 L, 3 L, 4 L, 5 L, 6 L, 7 L, 8 L, 9 L, 10 L, 25 L, 50 L, 100 L, 200 L, 300 L, 400 L, 500 L, 600 L, 700 L, 800 L, 900 L, or 1000 L, inclusive of all values and ranges falling within these volumes. In some embodiments, the chromatography vessel has a volume ranging from about 1 mL to about 10 mL, about 15 mL to about 50 mL, about 75 mL to about 300 mL, about 400 mL to about 900 mL, about 1 L to about 5 L, about 6 L to about 50 L, about 100 L to about 600 L, or about 700 L to about 1000 L.

In some embodiments, the ambient environment around the chromatography vessel is maintained (e.g., stabilized) at a temperature ranging from about 7° C. to about 13° C. In some embodiments, the ambient environment around the chromatography vessel is maintained at a temperature ranging from about 7° C. to about 13° C., from about 8° C. to about 12° C., from about 9° C. to about 11° C., from about 7° C. to about 12° C., from about 8° C. to about 11° C., or from about 9° C. to about 10° C. In some embodiments, the ambient environment around the chromatography vessel is maintained at a temperature of about 7° C., about 8° C., about 9° C., about 10° C., about 11° C., about 12° C., or about 13° C. In some embodiments, the ambient environment around the chromatography vessel is maintained at 10° C. In some embodiments, a thermal gradient of the ambient environment around the chromatography vessel may be used. For example, a negative thermal gradient may be established along a chromatography vessel such that the outlet end of the vessel is cooler than the inlet end of the vessel. Alternatively, a positive thermal gradient may be established along a chromatography vessel such that the outlet end of the vessel is warmer than the inlet end of the vessel.

In some embodiments, the temperature of the ambient environment around the chromatography vessel can be maintained at a desired temperature through the use of a temperature-controlled enclosure which is thermally coupled with a cooling supply or refrigeration device. Exemplary temperature-controlled enclosures include, but are not limited to, a jacket, a cooling block, a water bath, a cooling compartment or chamber, and a cooling tubing, that is thermally coupled with a cooling supply or refrigeration device. For example, in some embodiments, the temperature-controlled enclosure can be configured with circulated cooling gas or fluid (e.g., water). In some embodiments, the chromatography vessel is encased by a temperature-controlled jacket, which can be a water jacket or a gas jacket. In some embodiments, the temperature-controlled chromatography vessel is a water-jacketed chromatography vessel. In some embodiments, the temperature-controlled chromatography vessel is a gas-jacketed chromatography vessel. In some embodiments, the temperature-controlled chromatography vessel is a vacuum-jacketed for insulation.

In accordance with some embodiments, in order to maintain the temperature of the chromatography vessel at a desired temperature (in the range of about 7° C. to about 13° C.), the temperature-controlled enclosure (e.g., jacket) can be configured to encase at least a portion of the surface area of the chromatography vessel to ensure proper thermal transfer, for example at least 10%, 20%, 30%, 40%, 50%, 60% of the surface area of the chromatography vessel. In some embodiments, the temperature-controlled enclosure encases the entire chromatography vessel, however, in some other embodiments, the temperature-controlled enclosure covers a substantial portion of the surface area of the chromatography vessel, for example at least 50%, 60%, 70%, 80%, 90% of its surface area to ensure proper thermal transfer, and/or achieve a uniform temperature throughout the chromatography vessel. While a uniform thickness of the temperature-controlled enclosure can generally be used because its design and manufacture can be straightforward, the temperature-controlled enclosure can be configured to have a non-uniform thickness. For example, in some embodiments, the temperature-controlled enclosure may contain more gas or fluid (e.g., water) at the bottom portion than at the top portion. Alternatively, in some embodiments, the temperature-controlled enclosure may contain less gas or fluid (e.g., water) at the bottom portion than at the top portion. In addition or optionally, the temperature-controlled enclosure and the chromatography vessel can be manufactured (e.g., fabricated) as a single-component device.

### Temperature-Controlled Fluidic Channel

In some embodiments, the GCSF-containing sample is loaded onto the chromatography vessel via a fluidic channel, wherein the fluidic channel is placed in temperature-controlled enclosure or otherwise suitably equipped for temperature-controlled operations. In some embodiments, the ambient environment around the fluidic channel is maintained at a desired temperature. In some embodiments, the ambient environment around the fluidic channel is maintained at a temperature ranging from about 7° C. to about 13° C. In some embodiments, the ambient environment around the fluidic channel is maintained at a temperature ranging from about 7° C. to about 13° C., from about 8° C. to about 12° C., from about 9° C. to about 11° C., from about 7° C. to about 12° C., from about 8° C. to about 11° C., or from about 9° C. to about 10° C. In some embodiments, the ambient environment around the fluidic channel is maintained at a temperature of about 7° C., about 8° C., about 9° C., about 10° C., about 11° C., about 12° C., or about 13° C. In some embodiments, the ambient environment around the fluidic channel is maintained at 10° C. In some embodiments, a thermal gradient of the ambient environment around the fluidic channel may be used. For example, a negative thermal gradient may be established along a fluidic channel such that the outlet end of the vessel is cooler than the inlet end of the fluidic channel. Alternatively, a positive thermal gradient may be established along a fluidic channel such that the outlet end of the fluidic channel is warmer than the inlet end of the fluidic channel

In some embodiments, the temperature of the fluidic channel is maintained at a desired temperature through the use of a temperature-controlled enclosure such as, for example, a jacket, a cooling block, a water bath, a chamber, a compartment, or a tubing, which is thermally coupled with a cooling supply or refrigeration device. In some embodiments, the temperature-controlled enclosure can be configured with circulated cooling gas or fluid (water). In some embodiments, the fluidic channel is encased by a temperature-controlled jacket. In some embodiments, the temperature-controlled fluidic channel is a water-jacketed fluidic channel. In some embodiments, the temperature-controlled fluidic channel is a gas-jacketed fluidic channel. In some embodiments, the temperature-controlled fluidic channel is a vacuum-jacketed for insulation.

In accordance with some embodiments, in order to maintain the temperature of the fluidic channel at a desired temperature, the temperature-controlled enclosure (e.g., jacket) can be configured to encase at least a portion of the surface area of the fluidic channel to ensure proper thermal transfer, for example at least 10%, 20%, 30%, 40%, 50%, 60% of the surface area of the fluidic channel. In some embodiments, the temperature-controlled enclosure encases the entire fluidic channel, however, in some other embodiments, the temperature-controlled enclosure covers a substantial portion of the surface area of the fluidic channel, for example at least 50%, 60%, 70%, 80%, 90% of its surface area to ensure proper thermal transfer and/or achieve a uniform temperature throughout the chromatography vessel. While a uniform thickness of the enclosure can generally be used because its design and manufacture can be straightforward, the temperature-controlled enclosure can be configured to have a non-uniform thickness. For example, in some embodiments, the temperature-controlled enclosure may contain more gas or fluid (e.g., water) at the bottom portion than at the top portion. Alternatively, in some embodiments, the temperature-controlled enclosure may contain less gas or fluid (e.g., water) at the bottom portion than at the top portion. In addition or optionally, the temperature-controlled enclosure and the fluidic channel can be manufactured (e.g., fabricated) as a single-component device. In some embodiments, at least a portion of the fluidic channel is set at a temperature about 7° C. to about 13° C.

In some embodiments, the set temperatures of the enclosure of the vessel and the enclosure of the fluidic channel are the same. In some embodiments, the set temperatures of the enclosure of the chromatography vessel and the enclosure of the fluidic channel jacket are different. In some embodiments, at least one of the enclosures of the chromatography vessel and the enclosure of the fluidic channel is set at a temperature of about 7° C. to about 13° C. In some embodiments, at least one of the enclosures of the chromatography vessel and the enclosure of the fluidic channel is set at a temperature of about 10° C. In some embodiments, the temperature of the enclosure of the chromatography vessel is set at about 7° C. to about 13° C. and the temperature of the enclosure of the fluidic channel is set at room temperature (e.g., about 15° C. to about 25° C.). In some embodiments, the temperature of the enclosure of the chromatography vessel is set at about 10° C. and the temperature of the enclosure of the fluidic channel is set at room temperature (e.g., about 15° C. to about 25° C.).

In some embodiments, the chromatography vessel and the fluidic channel are placed in the same temperature-controlled enclosure. In other embodiments, the chromatography vessel and the fluidic channel are placed in separate temperature-controlled enclosures, such that the enclosure of the chromatography vessel and the enclosure of the fluidic channel can be set at different temperatures if desired.

In the methods disclosed herein, the fluidic channel can be any suitable fluid flow channel having a size dimension that enables a fluid flowing through. Generally, the fluidic channel may be a tube such as a flexible tube or a capillary tube, which is suitably equipped for temperature-controlled operations. In some embodiments, the fluidic channel can be a tube, a pipe, a bag, a container, a storage tank, a stirred-tank, a mixing vessel, or other fluid conduction means, which are suitably equipped for temperature-controlled operations. In some embodiments, the fluidic channel includes a temperature-controlled container which is configured to including a mixing mechanism, such as a static mixer or an ultrasonic mixer, to mix the GSCF-containing sample before and/or during loading. In some embodiments, the fluidic channel includes a temperature-controlled water-jacket. In some embodiments, the fluidic channel includes a temperature-controlled gas-jacket. In some embodiments, the fluidic channel includes a temperature-controlled vacuum jacket for insulation.

As discussed in greater detail below, embodiments of the methods disclosed herein include the use of one or more buffers. One skilled in the art will understand that the term buffer generally refers to a solution that resists changes in pH by the action of its acid-base conjugate component. Various buffers which can be employed depending, for example, on the desired pH of the buffer, the desired conductivity of the buffer, the characteristics of the GCSF protein to be purified, and the AEX materials. Determination of these parameters is well within the average skills of the person skilled in the art. Additional information in this regard can be found in, for example, “Buffers A guide for the preparation and use of buffers in biological systems” Mohan C., Calbiochem® Corp., 2006. Examples of buffers useful for the methods of the disclosure include loading buffers, equilibration buffers, elution buffers, and wash buffers. In some embodiments of the disclosure, one or more of the loading buffers, the equilibration buffers, elution buffers, and/or the wash buffers used during the purification process are the same. In some embodiments of the disclosure, all of the loading buffers, the equilibration buffers, elution buffers, and/or the wash buffers used during the purification process are the same. In some embodiments, the loading buffers, the equilibration buffers, elution buffers, and/or the wash buffers used during the purification process are different from one another.

In some embodiments, the method further including, prior to loading of the GCSF sample to be purified, equilibrating the anion exchange material with an equilibration buffer. Non-limiting examples of buffer systems suitable for the equilibration buffer of the disclosure include those based on phosphate, carbonate, borate, Tris, HEPES, MOPS, HEPPS, EPFS, CAPS, CAPSO, CHES, TES, BES, TAPS, Ethanolamine , Diethanolamine, Triethanolamine, Tricine, Bicine, Acetamidoglycine, Glycinamide or other biocompatible buffer substances having a pKa above 7. In some embodiments, the equilibration buffer includes Tris with a molarity within the range of about 30 mM to 50 mM, such as for example, about 30 mM to 40 mM, about 35 mM to 45 mM, about 40 mM to 50 mM, about 45 mM to 50 mM, about 30 mM to 45 mM, or about 35 mM to 50 mM. In some embodiments, the equilibration buffer includes Tris with a molarity of about 30 mM, 35 mM, 40 mM, 45 mM, or 50 mM. In some embodiments, the equilibration buffer includes Tris with a molarity of about 40 mM.

In some embodiments, suitable pH for the equilibration buffer ranges from about 7.0 to 8.0, such as for example, from about 7.0 to 7.5, about 7.1 to 7.6, about 7.2 to 7.7, about 7.3 to 7.8, about 7.4 to 7.9, or about 7.5 to 7.8. In some embodiments, the pH for the equilibration buffer ranges from about 7.4 to 7.8 or about 7.5 to 7.7. In some embodiments, the equilibration buffer has a pH of about 7.0, 7.1, 7.2, 7.3, 7.4, 7.5, 7.6, 7.7, 7.8, 7.9, or 8.0. In some embodiments, the equilibration buffer has a pH of about 7.5. In some embodiments, the equilibration buffer has a pH of about 7.6. In some embodiments, the equilibration buffer has a pH of about 7.7. In some embodiments, the equilibration buffer has a pH of about 7.8. In some embodiments, the equilibration buffer includes 40 mM Tris at pH of 7.6.

One of ordinary skill in the art will appreciate that the AEX material must be properly conditioned before use and also sufficiently equilibrated before loading of the GCSF-containing sample. For the methods disclosed herein, the AEX material can be equilibrated with at least 1 column volume (CV) of equilibration buffer. In some embodiments, the AEX material is equilibrated with at least 1 CV, 2 CV, 3 CV, 4 CV, 5 CV, 6 CV, 7 CV, or 8 CV of equilibration buffer. In some embodiments, the AEX material is equilibrated with 6 CV of equilibration buffer.

Prior to elution of the GCSF bound to the resin, the methods in accordance to some embodiments disclosed herein further include washing the anion exchange resin with a wash buffer, which is a buffer used to wash the chromatography material prior to eluting the GCSF. In some embodiments, the wash buffer is used to remove unbound or weakly bound contaminants. Examples of buffering agents suitable for use in the wash buffer include, but are not limited to, Tris, phosphate, carbonate, citrate, acetate, MOPS, HEPES, imidazole, and their salts or derivatives thereof. In some embodiments, the wash buffer includes Tris as a buffering agent. In some embodiments, the wash buffer includes Tris with a molarity within the range of about 30 mM to 50 mM, such as for example, about 30 mM to 40 mM, about 35 mM to 45 mM, about 40 mM to 50 mM, about 45 mM to 50 mM, about 30 mM to 45 mM, or about 35 mM to 50 mM. In some embodiments, the wash buffer includes Tris with a molarity of about 30 mM, 35 mM, 40 mM, 45 mM, or 50 mM. In some embodiments, the wash buffer includes Tris with a molarity of about 40 mM.

In some embodiments, suitable pH for the wash buffer ranges from about 7.0 to 8.0, such as for example, from about 7.0 to 7.5, about 7.1 to 7.6, about 7.2 to 7.7, about 7.3 to 7.8, about 7.4 to 7.9, or about 7.5 to 7.8. In some embodiments, the pH for the wash buffer ranges from about 7.4 to 7.8 or about 7.5 to 7.7. In some embodiments, the wash buffer has a pH of about 7.0, 7.1, 7.2, 7.3, 7.4, 7.5, 7.6, 7.7, 7.8, 7.9, or 8.0. In some embodiments, the wash buffer has a pH of about 7.5. In some embodiments, the wash buffer has a pH of about 7.6. In some embodiments, the wash buffer has a pH of about 7.7. In some embodiments, the wash buffer has a pH of about 7.8. In some embodiments, the wash buffer includes 40 mM Tris at pH of 7.6.

One skill in the art will appreciate that the wash buffer and the equilibration buffer can have different buffer compositions or have the same buffer composition. Accordingly, in some embodiments, wherein the wash buffer and the equilibration buffer have the same buffer composition. In some other embodiments, the wash buffer and the equilibration buffer have different buffer composition.

### Loading

When loading a GCSF sample onto a chromatography vessel containing a suitable AEX material, a loading buffer is used to load the sample which includes the GCSF and one or more impurities onto the chromatography vessel. In the bind-and-elute mode, the conductivity and/or pH of the loading buffer can be adjusted such that the GCSF is reversibly bound to the AEX chromatography material, while most, or ideally all, of the impurities are not bound and flow through the vessel.

Non-limiting examples of buffering agents suitable for use in the loading buffer include Tris, phosphate, carbonate, citrate, acetate, MOPS, HEPES, imidazole, and their salts or derivatives thereof. In some embodiments, the loading buffer includes Tris as a buffering agent. In some embodiments, the loading buffer includes Tris with a molarity within the range of about 30 mM to 50 mM, such as for example, about 30 mM to 40 mM, about 35 mM to 45 mM, about 40 mM to 50 mM, about 45 mM to 50 mM, about 30 mM to 45 mM, or about 35 mM to 50 mM. In some embodiments, the loading buffer includes Tris with a molarity of about 30 mM, 35 mM, 40 mM, 45 mM, or 50 mM. In some embodiments, the loading buffer includes Tris with a molarity of about 40 mM.

Suitable pH for the loading buffer ranges from about 7.4 to 8.0, such as for example, from about 7.4 to 7.6, about 7.5 to 7.7, about 7.6 to 7.8, about 7.7 to 7.9, or about 7.8 to 8.0. In some embodiments, the pH for the loading buffer ranges from about 7.4 to 7.8 or about 7.5 to 7.7. In some embodiments, the loading buffer has a pH of about 7.4, 7.5, 7.6, 7.7, 7.8, 7.9, or 8.0. In some embodiments, the loading buffer has a pH of about 7.5. In some embodiments, the loading buffer has a pH of about 7.6. In some embodiments, the wash buffer has a pH of about 7.7. In some embodiments, the loading buffer has a pH of about 7.8. In some embodiments, the loading buffer includes 40 mM Tris at pH of 7.6.

In some embodiments, wherein the loading buffer and the equilibration buffer have the same buffer composition. In some embodiments, the loading buffer and the equilibration buffer have different buffer composition. In instances where the loading buffer and the equilibration buffer have different buffer composition, before being loaded onto the chromatography vessel, the sample containing GCSF can be buffer exchanged into a buffer having similar composition to the equilibration buffer.

In addition to pH, the conductivity of the loading buffer can be adjusted such that the GCSF is reversibly bound to the AEX chromatography material, while most, or ideally all, of the impurities are not bound and flow through the vessel. Conductivity refers to the ability of an aqueous solution to conduct an electric current between two electrodes. In solution, the current flows by ion transport. Therefore, with an increasing amount of ions present in the aqueous solution, the solution will have a higher conductivity. The basic unit of measure for conductivity is the Siemen (or mho), mho (mS/cm), and can be measured using a conductivity meter, such as various models of Orion conductivity meters. Since electrolytic conductivity is the capacity of ions in a solution to carry electrical current, the conductivity of a solution may be altered by changing the concentration of ions therein. For example, the concentration of a buffering agent and/or the concentration of a salt (e.g., sodium chloride, sodium acetate, or potassium chloride) in the solution may be altered in order to achieve the desired conductivity. In some embodiments, the salt concentration of the loading buffer is modified to achieve the desired conductivity.

In some embodiments, the loading of the GCSF sample onto the chromatography vessel is performed at a conductivity below 3.0 mS/cm, such as below 2.5 mS/cm, below 2.0 mS/cm, below 1.5.0 mS/cm, or below 1.0 mS/cm. In some embodiments, the loading of the GCSF sample onto the chromatography vessel is performed at a conductivity ranging from about 1.0 to 2.0 mS/cm, from about 1.5 to 2.5 mS/cm, from about 2.0 to 3.0 mS/cm, from about 1.0 to 3.0 mS/cm, from about 1.5 to 3.0 mS/cm, or from about 1.0 to 2.5 mS/cm. In some embodiments, the loading of the GCSF sample onto the chromatography vessel is performed at a conductivity of about 0.5 mS/cm, 1.0 mS/cm, 1.5 mS/cm, 2.0 mS/cm, 2.5 mS/cm, or 3.0 mS/cm. In some embodiments, the loading of the GCSF sample onto the chromatography vessel is performed at a conductivity of about 2.5 mS/cm.

The purification methods disclosed herein can be used with GCSF-containing samples having a range of protein loading density, which generally refers to the amount of protein put in contact with a volume of chromatography material (e.g., AEX resin). Generally, loading density is expressed in g/L. In some embodiments, the GCSF-containing sample is loaded onto the chromatography vessel at a loading density of the GCSF ranging from about 1 g/L to 10 g/L, about 5 g/L to 15 g/L, about 10 g/L to 20 g/L, about 15 g/L to 25 g/L, or about 20 g/L to 30 g/L of the AEX material. In some embodiments, the GCSF-containing sample is loaded onto the chromatography vessel at a loading density of the GCSF of less than about any of 5 g/L, 10 g/L, 15 g/L, 20 g/L, 25 g/L, 30 g/L, 35 g/L, 40 g/L, 45 g/L, or 50 g/L of the AEX material. In some embodiments, the GCSF-containing sample is loaded onto the chromatography vessel at a loading density of the GCSF of less than about any of 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L of the AEX material. In some embodiments, the maximum loading density is 15 grams of GCSF per liter of AEX resin.

### Elution

Upon completion of loading the samples on the AEX chromatographic material and the GCSF product can be eluted (e.g., removed) from the AEX chromatographic material. For this purpose, an elution buffer can be used to elute the GCSF product from the solid phase, e.g., AEX chromatography material. In many cases, an elution buffer has a different physical characteristic than the load buffer and/or wash buffer. The conductivity and/or pH of the elution buffer can be adjusted such that the GCSF product is eluted from the AEX chromatography material. In some embodiments, the elution step is carried out under isocratic elution conditions in which the composition of the mobile phase is unchanged during the entire elution process. In some embodiments, the elution step is carried out under gradient elution (e.g., linear gradient elution) conditions in which the conductivity or pH in mobile phase is increased/decreased during the elution process. In some embodiments, the elution step is carried out under pseudo-gradient elution conditions in which two or more conditions are being altered during a gradient elution. An example of pseudo gradient method includes but is not limited to, increasing conductivity of the mobile phase while concurrently decreasing pH of the mobile phase during the elution process to achieve higher levels of purity when compared to performing an isocratic elution or a gradient that alters only one mobile phase condition. The combined effect of altering pH and salt concentration concurrently during ion exchange elution is another example of pseudo gradient elution.

In principle, the elution buffer can have any combination of higher or lower conductivity and higher or lower pH as compared to the load buffer and/or wash buffer. For example, the elution buffer may have a different conductivity than load buffer or a different pH than the load buffer. In some embodiments, the elution buffer has a lower conductivity than the load buffer. In some embodiments, the elution buffer has a higher conductivity than the load buffer. In some embodiments, the conductivity of the elution buffer changed from the conductivity of the load buffer and/or wash buffer by step gradient or by linear gradient. In some embodiments, the elution buffer has a lower pH than the load buffer. In some embodiments, the elution buffer has a higher pH than the load buffer. In some embodiments the elution buffer has a different conductivity and a different pH than the load buffer.

In addition, elution of the GCSF product from the chromatography material under bind-and-elute mode may be optimized for yield of product with minimal contaminants and at minimal pool volume. For example, the GCSF-containing sample may be loaded onto the AEX material, e.g. a chromatography column, in a load buffer. Upon completion of load, the GCSF product may be eluted with buffers at a number of different pH while the conductivity of the elution buffer is constant. Alternatively, the GCSF product may be eluted from the chromatography material in an elution buffer at a number of different conductivities while the pH of the elution buffer is constant. Upon completion of elution of the GCSF product from the chromatography material, the amount of contaminant in the pool fraction provides information regarding the separation of the product from the contaminants for a given pH or conductivity.

The elution buffer used to elute the GCSF from the solid phase, i.e., the AEX chromatography material. The conductivity and/or pH of the elution buffer can be monitored and/or adjusted to ensure that the GCSF bound to the AEX material is properly eluted. Non-limiting examples of buffer systems suitable for the elution buffer of the disclosure include those based on phosphate, carbonate, borate, Tris, HEPES, MOPS, HEPPS, EPFS, CAPS, CAPSO, CHES, TES, BES, TAPS, Ethanolamine, Diethanolamine, Triethanolamine, Tricine, Bicine, Acetamidoglycine, Glycinamide or other biocompatible buffer substances having a pKa above 7. In some embodiments, the elution buffer includes Tris with a molarity within the range of about 20 mM to 60 mM, such as for example, about 20 mM to 40 mM, about 25 mM to 45 mM, about 30 mM to 50 mM, about 35 mM to 55 mM, about 40 mM to 60 mM, about 20 mM to 50 mM, about 30 mM to 60 mM, or about 40 mM to 60 mM. In some embodiments, the equilibration buffer includes Tris with a molarity of about 30 mM, 35 mM, 40 mM, 45 mM, 50 mM, 55 mM, or 60 mM. In some embodiments, the equilibration buffer includes Tris with a molarity of about 30 mM to 60 mM Tris. In some embodiments, the elution buffer includes 40 mM Tris.

In some embodiments, the GCSF molecules that are bound to the AEX material can be eluted when the salt concentration in the elution buffer is increased. In some embodiments, the elution buffer includes sodium chloride (NaCl) at a molarity that can be adjusted to ensure that the GCSF bound to the AEX material is properly eluted. In some embodiments, the elution buffer includes NaCl with a molarity within the range of about 30 mM to 80 mM, such as for example, about 30 mM to 60 mM, about 35 mM to 65 mM, about 40 mM to 70 mM, about 45 mM to 75 mM, about 50 mM to 80 mM, about 30 mM to 50 mM, about 40 mM to 60 mM, or about 50 mM to 80 mM. In some embodiments, the equilibration buffer includes NaCl with a molarity of about 40 mM, 45 mM, 50 mM, 55 mM, 60 mM, 65 mM, 70 mM, 75 mM, or 80 mM. In some embodiments, the equilibration buffer includes NaCl with a molarity of about 60 mM to 70 mM NaCl. In some embodiments, the elution buffer includes 50 mM NaCl.

Suitable pH for the elution buffer ranges from about 7.4 to 8.0, such as for example, from about 7.4 to 7.6, about 7.5 to 7.7, about 7.6 to 7.8, about 7.7 to 7.9, or about 7.8 to 8.0. In some embodiments, the pH for the elution buffer ranges from about 7.4 to 7.8 or about 7.5 to 7.7. In some embodiments, the elution buffer has a pH of about 7.4, 7.5, 7.6, 7.7, 7.8, 7.9, or 8.0. In some embodiments, the elution buffer has a pH of about 7.5. In some embodiments, the elution buffer has a pH of about 7.6. In some embodiments, the elution buffer has a pH of about 7.7. In some embodiments, the elution buffer has a pH of about 7.8. In some embodiments, the elution buffer has at pH of 7.6.

In some embodiments, the elution of the GCSF from the AEX material is performed at a conductivity ranging from about 7.4 to 8.2 mS/cm, such as from about 7.4 to 8.0 mS/cm, about 7.5 to 8.1 mS/cm, or about 7.6 to 8.2 mS/cm. In some embodiments, the elution of the GCSF from the AEX material is performed at a conductivity of about 7.4 mS/cm, 7.5 mS/cm, 7.6 mS/cm, 7.7 mS/cm, 7.8 mS/cm, 7.9 mS/cm, 8.0 mS/cm, 8.1 mS/cm, or 8.2 mS/cm. In some embodiments, the elution of the GCSF from the AEX material is performed at a conductivity of about 7.8 mS/cm.

In some embodiments of the disclosed methods, chromatographic fractions of the eluted product (e.g., eluate) can be collected. In some embodiments, the chromatographic fractions collected are greater than about 0.01 CV (column volume), 0.02 CV, 0.03 CV, 0.04 CV, 0.05 CV, 0.06 CV, 0.07 CV, 0.08 CV, 0.09 CV, 0.1 CV, 0.2 CV, 0.3 CV, 0.4 CV, 0.5 CV, 0.6 CV, 0.7 CV, 0.8 CV, 0.9 CV, 1.0 CV, 2.0 CV, 3.0 CV, 4.0 CV, 5.0 CV, 6.0 CV, 7.0 CV, 8.0 CV, 9.0 CV, or 10.0 CV. In some embodiments, chromatographic fractions of 0.5 CV in volume were collected. In some embodiments, chromatographic fractions containing the product, e.g. GCSF, are pooled. In some embodiments, fractions containing the GCSF from the load fractions and from the elution fractions are pooled. The amount of GCSF in a chromatographic fraction can be determined by one skilled in the art; for example, the amount of GCSF in a chromatographic fraction can be determined by UV spectroscopy using absorbance at 280 nm, or by Coomassie dye. In some embodiments, fractions containing detectable GCSF amount are pooled. In some embodiments, the AEX material may be stripped, e.g. after elution, with high salt concentration (e.g., 5 CVs of 2 M NaCl). In some embodiments, the method further includes a sanitizing step of the AEX material with, e.g., with 5 CVs 0.5 N sodium hydroxide, or with 2 M NaCl, 0.5 N NaOH.

In some embodiments, the DEAE chromatography is operated at a linear flow rate for all phases. In some embodiments, the linear flow rate is less than about any of 50 CV/hr, 40 CV/hr, or 30 CV/hr. The flow rate may be between about any of 5 CV/hr and 50 CV/hr, 10 CV/hr and 40 CV/hr, or 18 CV/hr and 36 CV/hr. In some embodiments, the flow rate is about any of 9 CV/hr, 18 CV/hr, 25 CV/hr, 30 CV/hr, 36 CV/hr, or 40 CV/hr. In some embodiments, the flow rate is less than about any of 200 cm/hr, 175 cm/hr, 150 cm/hr, 125 cm/hr, 100 cm/hr, 75 cm/hr, 50 cm/hr, or 25 cm/hr. The flow rate may be between about any of 25 cm/hr and 200 cm/hr, 50 cm/hr and 150 cm/hr, 100 cm/hr and 175 cm/hr, 50 cm/hr and 100 cm/hr, 125 cm/hr and 175 cm/hr, 100 cm/hr and 200 cm/hr, or 250 cm/hr and 200 cm/hr. In some embodiments, the linear flow rate is about 150 cm/hr. In some embodiments, at least one of the strip and sanitization phases is performed at 25 cm/hr, 50 cm/hr, or 75 cm/hr. In some embodiments, at least one of the strip and sanitization phases is performed at 50 cm/hr. In some embodiments, the strip and sanitization phases are performed at different flow rates. In some embodiments, the strip and sanitization phases are performed at the same low rate.

### GCSF Sources

As discussed above, the GCSF protein, e.g., human GCSF, to be purified using the methods described herein is generally produced using recombinant techniques in eukaryotic organisms (e.g., yeast and mammalian cell lines) or in prokaryotic organism such as bacteria (e.g., E. coli). When using recombinant techniques, the GCSF protein can be produced intracellularly, in the periplasmic space, or directly secreted into the medium. The form of recombinant GCSF is produced depends on the type of host organism used for expression. When the recombinant GCSF is expressed in eukaryotic cells, it is generally produced in a soluble form and secreted. On the other hand, the expression of a recombinant GCSF in prokaryotic cells, e.g. E. coli, often results in the formation of inactive and insoluble aggregates, called inclusion bodies (IBs), which generally have a secondary structure and are densely aggregated. In existing purification processes, these insoluble aggregates are generally solubilized in a buffer containing denaturing agents, followed by a renaturation step to transform the denatured forms into biologically active forms with apposite three-dimensional structure. Accordingly, in some embodiments, the GCSF-containing sample includes IBs that are obtained from a recombinant cell expressing GCSF wherein the expressed GCSF forms the IBs in the recombinant cell. In some embodiments, the recombinant cell is a prokaryotic cell. In some embodiments, the prokaryotic cell is an E. coli cell. In some embodiments, the recombinant cell is a eukaryotic cell. In some embodiments, the recombinant eukaryotic cell is a fungal cell (e.g., filamentous fungus or yeast), an insect cell, such as an SF9 cell, or an animal cell. In some embodiments, the animal cell is recombinant mammalian cell, such as a CHO cell, BHK cell, HEK cell, e.g. HEK 293 cell. In some embodiments, the recombinant fungal cell is a Saccharomyces cerevisiae cell or a Pichia pastoris cell.

The recombinantly produced GCSF protein may be recovered from culture medium or from host cell lysates. Cells employed in expression of the polypeptides can be disrupted by various physical or chemical means, such as freeze-thaw cycling, sonication, mechanical disruption, or cell lysing agents. If the polypeptide is produced intracellularly, as a first step, the particulate debris, either host cells or lysed fragments, are removed, for example, by centrifugation or ultrafiltration. Cell debris can be removed by centrifugation. Where the GCSF is secreted into the medium, supernatants from such expression systems are generally first concentrated using a commercially available polypeptide concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Accordingly, in some embodiments, the GCSF-containing sample can contain human GCSF (hGCSF) polypeptide. In some embodiments, the GCSF polypeptide can be a polypeptide having the sequence of human granulocyte colony stimulating factor (hGCSF) as shown in SEQ ID NO: 1, or a variant of hGCSF that exhibits GCSF activity.

(SEQ ID NO: 1)
MTPLGPASSLPQSFLLKCLEQVRKIQGDGAALQEKLCATYKLCHPEEL
VLLGHSLGIPWAPLSSCPSQALQLAGCLSQLHSGLFLYQGLLQALEGI
SPELGPTLDTLQLDVADFATTIWQQMEELGMAPALQPTQGAMPAFASA
FQRRAGGVLVASHLQSFLEVSYRVLRHLAQP.

The GCSF activity can be assessed by its ability of the GCSF polypeptide to bind to a GCSF receptor in vivo or ex vivo. The GCSF activity can also be its cell-proliferation activity, which can be determined in, for example, an in vitro activity assay using the murine cell line NFS-60 (ATCC CRL-1838). A suitable in vitro assay for GCSF activity using the NFS-60 cell line is described in by Hammerling et al. in J. Pharm. Biomed. Anal. 13 (1):9-20, 1995. A polypeptide exhibiting GCSF activity is considered to have such activity when it displays a measurable function, e.g. a measurable proliferative activity in the in vitro assay. Functional activity of GCSF can also be determined by assaying one or more of its effects in (i) growth and migration of endothelial cells, (ii) decrease of norepinephrine reuptake, (iii) increase in osteoclastic activity, and (iv) decrease in osteoblast activity. Functional activity of GCSF can also be determined by its ability to promote the differentiation and proliferation of hematopoietic precursor cells, and/or the activation of mature cells of the hematopoietic system.

In some embodiments, the term “GCSF variant” generally refers to a polynucleotide differing from the hGCSF polypeptide, but retaining essential properties thereof. Generally, GCSF variants are overall closely similar, and, in many regions, identical to the hGCSF polypeptide. As such, the term GCSF variant encompasses polypeptides having amino acid sequences that are at least 80%, at least 85%, at least 90%, at least 95%, at least 96%, at least 97%, at least 98%, or at least 99% identical to the amino acid sequence of a human GCSF. In some embodiments, a variant refers to a polypeptide having amino acid sequences that are at least 80%, at least 85%, at least 90%, at least 95%, at least 96%, at least 97%, at least 98%, or at least 99% identical to the amino acid sequence of SEQ ID NO: 1:

The term “functional variant” refers to polypeptide variants that are fully functional in comparison to a hGCSF; or which retain at least some, for example at least 50%, at least 60%, at least 70%, at least 80%, at least 90%, at least 95%, at least 96%, at least 97%, at least 98%, or at least 99% of the biological activity of a hGCSF. In some embodiments, a functional variant refers to polypeptide variants that are fully functional in comparison to the polypeptide of SEQ ID NO: 1. As such, the term functional variant encompasses a functional fragment, derivative (e.g., structurally and functionally similar to hGCSF. In some embodiments, functional variant of can be a fusion molecule or fusion protein thereof. It is understood that polypeptides, fusion proteins, fusion molecules and protein complexes coupled with the polypeptides or functional polypeptide variants are also encompassed by the term “functional variant.” In some embodiments, a hGCSF functional variant of the present disclosure is capable of promoting the differentiation and proliferation of hematopoietic precursor cells, and/or the activation of mature cells of the hematopoietic system.

In some embodiments of the method disclosed herein, the loading sample includes GCSF produced by eukaryotic recombinant cells such as, insect cells or CHO cell, and subsequently secreted to the culture medium. In some embodiments, the loading sample includes GCSF obtained from insoluble IBs. Yet in some embodiments, the loading sample includes GCSF obtained from both sources, i.e. from secreted GCSF and GCSF obtained from insoluble IBs.

### Additional Purification Techniques

To ensure the safety of biopharmaceuticals, regulatory agencies impose stringent purification standards and relevant quality attributes (e.g., identity, purity, and biological activity) for recombinant proteins intended for human administration. In order to obtain GCSF product of higher quality, some embodiments of the methods may further include one or more purification steps either prior to, or after, any of the AEX chromatography described herein. Generally, any protein purification techniques known in the art can be used in combination of the purification methods disclosed herein. Examples of protein purification techniques suitable for use in the methods of the disclosure include, but are not limited to, ion exchange chromatography such as AEX and cation exchange chromatography (CEX), affinity chromatography such as protein A chromatography and protein G chromatography, mixed mode chromatography (MMC), hydroxyapatite chromatography, gel filtration chromatography; affinity chromatography, gel electrophoresis, dialysis, ethanol precipitation, reverse phase HPLC, chromatography on silica, chromatofocusing, SDS-PAGE, ammonium sulfate precipitation, and metal chelating columns to bind epitope-tagged forms of GCSF. In some embodiments, the methods disclosed herein may further include one or more purification steps selected from affinity chromatography, CEX chromatography, hydroxyapatite chromatography, size exclusion chromatography (SEC), hydrophobic interaction chromatography (HIC), metal affinity chromatography, and MMC chromatography. Non-chromatography separation techniques can also be considered, such as precipitation with salt, acid, or with a polymer PEG. Additional non-chromatography protein purification techniques suitable for use in the methods of the disclosure include, but are not limited to, centrifugation, extraction, diafiltration, and ultrafiltration.

In some embodiments, the one or more protein purification techniques includes cation exchange chromatography (CEX). In some embodiments, the methods of the disclosure further a CEX step performed with a selected material (e.g., CM Sepharose® FF) that allows particularly high flow rates and good product recovery. In these instances, due to the fact that it is positively charged in an acidic environment, GCSF is a strong binder and can be eluted with a linear sodium chloride gradient at an acidized pH in a small volume at a high concentration in the desired buffer. Systems and methods for performing cation exchange chromatography are well known to the person skilled in the art. Generally, the GCSF binds to the cation exchange matrix within a specific pH range due to its positive total charge, while most of the contaminating substances like nucleic acids, lipopolysaccharides and proteins originating from host cells as well as ionic isomers of GCSF and altered forms of GCSF having different pH values are not capable of binding and appear in the flow-through or are of being removed by means of washing.

Suitable functional groups used for CEX resins include, but are not limited to, carboxymethyl (CM), sulfonate (S), sulfopropyl (SP) and sulfoethyl (SE). These are commonly used cation exchange functional groups for biochromatographic processes. Suitable commercially available products include, but are not limited to, carboxymethyl (CM) cellulose, AG 50 W, Bio-Rex 70, carboxymethyl (CM) Sephadex, sulfopropyl (SP) Sephadex, carboxymethyl (CM) Sepharose CL-6B, CM Sepharose HP, Hyper D-S ceramic (Biosepra) and sulfonate (S) Sepharose, SP Sepharose FF, SP Sepharose HP, SP Sepharose 15 XL, CM Sepharose FF, TSK gel SP SPW, TSK gel SP-SPW-HR, Toyopearl SP-650M, Toyopearl SP-650S, Toyopearl SP-650C, Toyopearl CM-650M, Toyopearl CM-650S etc. Sulfopropyl matrices, in particular the products SP Sepharose XL and SP Sepharose FF (Fast Flow) and S-Sepharose FF. In some embodiments, the cation exchange material is a sulfopropyl cation exchange material. In some particular embodiments, the CEX is performed with CM-Sepharose FF.

In some embodiments, the one or more protein purification techniques includes an ultra-, micro- or diafiltration operation to remove contaminants such as cell debris, insoluble contaminating proteins, and nucleic acid precipitates. This filtration operation provides a suitable means to economically remove cell debris, contaminating proteins and precipitate. It will be appreciated by one of ordinary skill in the art that in choosing a filter or filter scheme, it is important to ensure a robust performance in the event upstream changes or variations occur. Maintaining the balance between good clarification performance and step yield requires investigation of a large variety of filter types with various filter media. Suitable filter types can include cellulose filters, regenerated cellulose fibers, cellulose fibers combined with inorganic filter aids (e.g., diatomaceous earth, perlite, fumed silica), cellulose fibers combined with inorganic filter aids and organic resins, or any combination thereof, and polymeric filters to achieve effective removal. Suitable examples of polymeric filters include, but are not limited to nylon, polypropylene, polyether sulfone. In some embodiment, the filtration operation, e.g., the diafiltration and/or ultrafiltration step is performed using a polyether sulfone membrane. In some embodiment, the diafiltration and/or ultrafiltration step is performed using a Sius Hystream® membrane.

In addition, in some embodiments disclosed herein, in order to achieve higher product concentration of the GCSF preparation, the GCSF protein can be dialyzed, ultrafiltrated, or diafiltrated to remove contaminants such as unwanted buffer components. In particular, diafiltration is a fractionation process of washing smaller molecules through a membrane, leaving the larger molecule of interest in the retentate. It is widely considered a suitable and efficient technique for removing or exchanging salts, removing detergents, separating free from bound molecules, removing low molecular weight materials, or rapidly changing the ionic or pH environment. The diafiltration process generally employs a microfiltration or an ultrafiltration membrane in order to remove a product of interest from slurry while maintaining the slurry concentration as a constant.

In some embodiments, at least one additional purification process as described above can be performed prior to the AEX chromatography process. In some embodiments, at least one additional purification process as described above can be performed after the AEX chromatography process.

Another aspect of this application relates to a system for manufacturing GCSF including a chromatography vessel containing an AEX material capable of binding GCSF, in which the chromatography vessel is encased in a temperature-controlled enclosure and at least a portion of the enclosure of the chromatography vessel is set a temperature of about 7° C. to about 13° C. In some embodiments, the system further includes a fluidic channel placed in a temperature-controlled enclosure, wherein at least a portion of enclosure of the fluidic channel is set at a temperature about 7° C. to about 13° C.

### Compositions of the Disclosure

The present disclosure also provides GCSF molecules purified by the methods disclosed herein, as well as pharmaceutical compositions containing the same. In some embodiments, the purified GCSF obtained by such methods can be biologically active GCSF with improved purity and/or functional activity, and can be particularly suited for therapeutic applications.

In some embodiments, the purified GCSF contains biologically active GCSF with a purity of greater than 80%. In some embodiments, the purified GCSF includes biologically active GCSF with a purity of greater than 85%, 90%, 95%, 96%, 97%, 98%, or 99%. Various methods for quantifying the degree of purification of the purified GCSF are known to those of skill in the art. These include, for example, determining the specific activity of an active GCSF, or assessing the amount of GCSF in the end product by SDS-PAGE analysis. An exemplary method for assessing the purity of a GCSF obtained from the disclosed method is to calculate the specific activity of the obtained GCSF, and to compare it to the specific activity of the initial extract, and to thus calculate the degree of purity.

The biological activity of the GCSF obtained according to the present disclosure can be determined by a number of techniques known in the art, for example, by means of a bioassay known in the art and compared with the activity of a standard, commercially available GCSF. For example, biological activity of the GCSF obtained from the method as disclosed herein can be determined by an assay based on stimulation of cellular proliferation (NFS-60 cells) using the method described by Hammerling et al. (1995, supra)) and the use of an international standard human recombinant GCSF.

It can then be shown that cells treated with the GC SF purified as described herein grow just as well or better as those cells that are treated with the standard. In particular, it can be shown that purified GCSF obtained according to the method of the present disclosure is characterized by a biological activity of 80-100% referring to WHO-reference standard in the NFS-60 proliferation assay.

In some embodiments, the GCSF purified as described herein is GCSF with improved purity and/or functional activity. In some embodiments, the GCSF as described herein has a purity of greater than 80% such as, for example, a purity of greater than 85%, 90%, 95%, 96%, 97%, 98%, or 99%. In some embodiments, the obtained GCSF exhibits a significant increase in specific activity, for example, a specific activity of at least 1×10⁵ IU/mg. In some embodiments, the obtained GCSF has a specific activity of at least 1×10⁶ IU/mg, preferably at least 1×10⁷ IU/mg, more preferably within a range of specific activity 2-9×10⁷ IU/mg, and most preferably a specific activity of about 1×10⁸ IU/mg, wherein the specific activity is measured by a method based on stimulation of cellular proliferation.

In a related aspect of the disclosure, some embodiments disclosed herein relate to a pharmaceutical composition which includes a therapeutically effective amount of the biologically active GCSF as disclosed herein and is suitable for therapeutic and clinical use.

The pharmaceutical compositions in accordance with the disclosure include compositions and formulations for human and veterinary use. In some embodiments, the pharmaceutical composition includes a mixture of the biologically active GCSF as disclosed herein with a pharmaceutically acceptable auxiliary substance. Suitable pharmaceutically acceptable auxiliary substances include suitable diluents, adjuvants and/or carriers useful in GCSF therapy. Non-limiting examples of pharmaceutically acceptable auxiliary substance include, but is not limited to, saline, solvents, dispersion media, coatings, antibacterial and antifungal agents, isotonic and absorption delaying agents, and the like, compatible with pharmaceutical administration. Supplementary active substances can also be incorporated into the compositions. In some embodiments, the pharmaceutical composition further includes pharmaceutically acceptable additives such as buffers, salts and stabilizers. The GCSF and the pharmaceutical compositions obtained according to the present disclosure can either be (i) used directly or (ii) further processed, for instance pegylated as described in greater detail below or in, e.g., PCT Publication No. WO2008/124406 and then stored in the form of a powder or a lyophilisate or in liquid form. In some embodiments, the pharmaceutical composition of the disclosure is a liquid composition. In some embodiments, the pharmaceutical composition of the disclosure is a lyophilisate or a powder.

The GCSF as an active ingredient of a pharmaceutical composition can be administered in a typical method through an intravenous, intra-arterial, intraperitoneal, intrastemal, transdermal, nasal, inhalant, topical, rectal, oral, intraocular or subcutaneous route. The administration method is not particularly limited, but a non-oral administration is preferable, and the subcutaneous or intravenously administration is more preferable.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration, suitable auxiliary substance include physiological saline, bacteriostatic water, Cremophor EL™ (BASF, Parsippany, N.J.) or phosphate buffered saline (PBS). In some cases, the composition should be sterile and should be fluid to the extent that easy syringability exists. It should be stable under the conditions of manufacture and storage and must be preserved against the contaminating action of microorganisms such as bacteria and fungi. The auxiliary substance can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Additional examples of suitable adjuvants in the pharmaceutical compositions containing GCSF as disclosed herein include, but are not limited to, stabilizers like sugar and sugar alcohols, amino acids and tensides like for example Polysorbate-20, Polysorbate-60, Polysorbate-65, Polysorbate-80, as well as suitable buffer substances. In some embodiments according to the methods of the present disclosure, the purified GCSF is formulated in 10 mM acetic acid at a pH of 4.0, 0.0025% Polysorbate 80 and 50 g/L Sorbitol.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the common methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., GCSF disclosed herein and/or pharmaceutical compositions containing the same) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches, and the like, can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject GCSF and/or pharmaceutical compositions as disclosed herein of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the subject GCSF and/or pharmaceutical compositions as disclosed herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the subject GCSF and/or pharmaceutical compositions as disclosed herein can also be prepared in the form of suppositories (e.g., with conventional suppository bases such as cocoa butter and other glycerides) or retention enemas for rectal delivery. In some embodiments, the GCSF and/or pharmaceutical compositions of the disclosure can also be administered by transfection or infection using methods known in the art.

In one embodiment, the pharmaceutical compositions of the disclosure are prepared with carriers that will protect the recombinant GCSF against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the recombinant GCSF of the disclosure can be further modified to prolong their half-life in vivo and/or ex vivo. Non-limiting examples of known strategies and methodologies suitable for modifying the recombinant GCSF of the disclosure include (1) chemical modification of a polypeptide described herein with highly soluble macromolecules such as polyethylene glycol (“PEG”) which prevents the polypeptides from contacting with proteases; and (2) covalently linking or conjugating a polypeptide described herein with a stable protein such as, for example, albumin. Accordingly, in some embodiments, the recombinant GCSF of the disclosure can be fused to a stable protein, such as, albumin. For example, human albumin is known as one of the most effective proteins for enhancing the stability of polypeptides fused thereto and there are many such fusion proteins reported.

In some embodiments, the recombinant GCSF of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the interferon. In some embodiments, the PEGylated or PASylated GCSF polypeptide contains a PEG or PAS moiety on only one amino acid. In other embodiments, the PEGylated or PASylated GCSF polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated GCSF polypeptide may be coupled directly to PEG or PAS (e.g., without a linking group) through an amino group, a sulfhydryl group, a hydroxyl group, or a carboxyl group. In some embodiments, the recombinant GCSF of the disclosure is covalently bound to a polyethylene glycol with an average molecular weight of 20,000 Daltons.

In some embodiments, the pharmaceutical compositions of the disclosure include one or more pegylation reagent. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. Examples of pegylation reagents suitable for the methods and compositions of the disclosure include, but are not limited to, methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol. In some embodiments, the pegylation reagent is polyethylene glycol with an average molecular weight of 20,000 Daltons covalently bound to the N-terminal methionine residue of the protein.

### Methods of Treatment

The purified GCSF obtained in accordance with the methods of the present disclosure, and particularly the biologically active GCSF obtained by such methods, can be particularly suited for therapeutic applications. Accordingly, also provided in some embodiments of the disclosure are methods for treating or preventing a disease or health condition in a subject in need thereof, the methods including administering to the subject a therapeutically effective amount of a GCSF as disclosed herein and/or a pharmaceutical composition as disclosed herein.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering. The term “therapeutically effective amount” used herein refers to the amount of biologically active GCSF obtained by the methods disclosed herein which has the therapeutic effect of biologically active GCSF.

In some embodiments, the GCSF and/or pharmaceutical composition as disclosed herein is formulated to be compatible with its intended route of administration. The GCSF and/or pharmaceutical composition of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject GCSF and/or pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices can be commonly used. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in mammals, e.g., humans. The dosage of such compounds lies generally within a range of circulating concentrations that include the ED₅₀ with little or no toxicity. The dosage may vary within this range depending upon the dosage form employed and the route of administration utilized. For any pharmaceutical compositions used in the treatment methods of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, the methods of the disclosure are suitable for the treatment and/or prevention of a disease or health condition associated with one or more indications selected from the group consisting of neutropenia and neutropenia-related clinical sequelae, chronic neutropenia, neutropenic and non-neutropenic infections, reduction of hospitalization for febrile neutropenia after cytotoxic chemotherapy and for the reduction in the duration of neutropenia in patients undergoing myeloablative therapy followed by bone marrow transplantation considered to be at increased risk of prolonged severe neutropenia. In some embodiments, the methods of the disclosure are suitable for the treatment and/or prevention of a disease or health condition associated with the mobilization of peripheral blood progenitor cells (PBPC) and chronic inflammatory conditions.

In some embodiments, long term administration of the GCSF disclosed herein and/or pharmaceutical compositions containing the same is indicated to increase neutrophil counts and to reduce the incidence and duration of infection-related events, treatment of persistent neutropenia in patients with advanced HIV infection, in order to reduce the risk of bacterial infections. In some embodiments, the GCSF disclosed herein and/or pharmaceutical compositions containing the same is indicated for improving the clinical outcome in intensive care unit patients and critically ill patients, wound/skin ulcers/burns healing and treatment, intensification of chemotherapy and/or radiotherapy, increase of anti-inflammatory cytokines, potentiation of the antitumor effects of photodynamic therapy. In some embodiments, the GCSF disclosed herein and/or pharmaceutical compositions containing the same is indicated for prevention and treatment of illness caused by different cerebral dysfunctions, treatment of thrombotic illness and their complications and post irradiation recovery of erythropoiesis. It can be also used for treatment of all other illnesses reported as indicative for GCSF.

A pharmaceutical composition containing the biologically active GCSF obtained by the methods disclosed herein can thus be administered, to patients, children or adults in a therapeutically amount which is effective to treat or prevent one or more of the above mentioned disease or health conditions. In some embodiments, the methods of the disclosure are suitable for the treatment and/or prevention of neutropenia.

As discussed above, any one of the compositions as disclosed herein, e.g., purified GCSF and pharmaceutical compositions, can be administered to a subject in need thereof as a single therapy (e.g., monotherapy). In addition or alternatively, in some embodiments of the disclosure, one or more of the purified GCSF and pharmaceutical compositions described herein can be administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Systems

Also provided herein are various systems for manufacturing GCSF. In particular, some embodiments of the disclosure relate to systems including one or more chromatography vessels including an anion exchange chromatography (AEX) material capable of binding GCSF, wherein the chromatography vessel is encased in a temperature-controlled enclosure and at least a portion of the enclosure of the chromatography vessel is set a temperature of about 7° C. to about 13° C. In some embodiments, the systems described herein include an upstream component including a bioreactor and a downstream component including a purification module with one or more AEX chromatography vessels as described herein. In some embodiments, the bioreactor includes a growth chamber containing a suspension with at least one cell culture medium and at least one recombinant cell configured to express GCSF.

Non-limiting exemplary embodiments of the disclosed systems include one or more of the following features. In some embodiments, the system further includes a fluidic channel placed in a temperature-controlled enclosure, wherein at least a portion of enclosure of the fluidic channel is set at a temperature 7° C. to about 13° C. In some embodiments, the set temperatures of the enclosure of the vessel and the enclosure of the fluidic channel are the same. In some embodiments, the set temperatures of the enclosure of the chromatography vessel and the enclosure of the fluidic channel jacket are different. In some embodiments, at least one of the enclosure of the chromatography vessel and the enclosure of the fluidic channel is set at a temperature of about 10° C. In some embodiments, the chromatography vessel is selected from the group consisting of a column, a tank, a packed bed, a fluidized bed, a cartridge, an encapsulated membrane, a reservoir, a chamber, a container, and a mixing vessel. In some embodiments, the fluidic channel is a tube, a pipe, a bag, a container, a storage tank, a mixing vessel, or other fluid conduction means.

In some embodiments, prior to loading of the GCSF sample, the AEX material is equilibrated with an equilibration buffer including from about 30 mM to about 50 mM Tris, and at pH of about 7.0 to about 8.0. In some embodiments, the equilibration buffer includes about 40 mM Tris and at pH of about 7.6. In some embodiments, prior to elution of the GCSF, the AEX material is washed with a wash buffer to remove unbound or weakly bound contaminants. In some embodiments, the wash buffer and the equilibration buffer have the same buffer composition. In some embodiments, the GCSF-containing sample includes a loading buffer. In some embodiments, the loading buffer has a pH of about 7.4 to about 8.0. In some embodiments, the loading of the GCSF sample onto the chromatography vessel is carried out at a conductivity ranging between about 1.5 to about 3.0 mS/cm.

In some embodiments of the disclosed systems, the elution buffer includes about 30 mM-60 mM Tris, about 30 mM-80 mM sodium chloride, and a pH of about 7.4 to about 8.0. In some embodiments, the elution buffer includes about 40 mM Tris, about 50 mM sodium chloride, and pH of about 7.7. In some embodiments, the elution of the GCSF from the AEX material is carried out at a conductivity ranging between about 7.4 to about 8.2 mS/cm. In some embodiments, the AEX material includes diethylaminoethyl (DEAE) ion-exchange chromatography. In some embodiments, the AEX material includes DEAE Sepharose® resin. In some embodiments, the DEAE Sepharose® resin includes DEAE Sepharose® Fast Flow resin.

In some embodiments, the systems further includes one or more phases of stripping and/or sanitation of the AEX material. In some embodiments, the DEAE chromatography is operated at a linear flow rate for all phases. In some embodiments, the linear flow rate is about 150 cm/hr. In some embodiments, at least one of the stripping and sanitization phases is performed at 50 cm/hr. In some embodiments, the GCSF is a recombinant human GCSF (hGCSF) or a variant thereof. In some embodiments, the sample includes GCSF obtained from a recombinant eukaryotic cell or a recombinant prokaryotic cell.

In some embodiments, the systems disclosed herein further include at least one additional purification process. In some embodiments, the at least one additional purification process is selected from the group consisting of affinity chromatography, cation exchange chromatography (CEX), hydroxyapatite chromatography, size exclusion chromatography (SEC), hydrophobic interaction chromatography (HIC), metal affinity chromatography, mixed mode chromatography (MMC), centrifugation, diafiltration, and ultrafiltration. In some embodiments, the at least one additional purification process is performed prior to the AEX chromatography process. In some embodiments, the at least one additional purification process is performed after the AEX chromatography process.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventor reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

Additional embodiments are disclosed in further detail in the following examples, which are provided by way of illustration and are not in any way intended to limit the scope of this disclosure or the claims.

### Example 1

### Improvement of Anion Exchange Chromatography Parameters

This Example summarizes the results of experiments performed to evaluate the influence of a number of AEX chromatographic parameters on GCSF product-related quality and yield, including: (1) AEX operation temperature, (2) load density, (3) fractionation, and (4) resin lifetime. As described in greater detail below, in preliminary hold time stability studies performed with DEAE Sepharose® Fast Flow resin, yield instability was observed when the DEAE load was maintained under room temperature conditions.

### Column Operation Temperature

Due to the need to store the DEAE load at a chilled temperature for stability, DEAE chromatography operations were evaluated with a load that was chilled in an ice water bath (2-8° C.). In addition, to understand the impact of temperature on the chromatography itself, a chilled chromatography column (in an ice water bath, 2-8° C.) was also evaluated. As summarized in TABLE 1, four experimental conditions were tested: (1) cold load and cold column; (2) room temperature load and cold column; (3) cold load and room temperature column; and (4) room temperature load and room temperature column. The same feedstock was used for all four conditions. When needed, an ice bath was used to chill the column and the load. Based upon the data in Table 1, it was observed that the chilled column offered the greatest improvement to yield.

TABLE 1
Temperature conditions of DEAE operation.

| Process Condition | | DEAE |
|---|---|---|
| Load | Column | (% Yield) |
| Cold | Cold | 72.9 |
| Room Temp | Cold | 74.3 |
| Cold | Room Temp | 50.6 |
| Room Temp | Room Temp | 45.6 |

Subsequently, a more controlled temperature study was performed with GCSF sample from Experiment 1. This material was loaded on DEAE column at a loading density of 15 g/L, with both the load and column being chilled by one chiller at equivalent temperature set-points of 10° C., 15° C., and 20° C. A summary of the results from this controlled temperature study is presented in TABLE 2.

TABLE 2
Controlled temperature DEAE operations.

| Process Condition | | DEAE |
|---|---|---|
| Load | Column | (% Yield) |
| 10° C. | 10° C. | 49.8 |
| 15° C. | 15° C. | 41.2 |
| 20° C. | 20° C. | 33.8 |

It was observed that lower temperature offered improvement in yield although yield still varied between the feedstocks processed to generate data presented in TABLE 1 and TABLE 2. Without being bound to any particular theory, this variation in yield was most likely due to changes in the feedstock product quality over time (e.g., feedstock age). Based upon the data presented in TABLE 1 and TABLE 2, in future GCSF production in accordance to GMP regulation, a temperature-controlled equipment having a jacketed column and load vessel was implemented. The temperature set-point of 10° C. was configured for the jacket of this equipment.

### Load Density

The effects of load density on DEAE chromatography was evaluated with two different feedstocks. In the first evaluation, a GCSF-containing sample (Sample 1) was loaded onto a DEAE column with increasing load densities: 10 g/L, 15 g/L, 20 g/L, and 25 g/L. In this first study, the load was chilled at 10° C. and the column was maintained at room temperature. The GCSF yield obtained in these load density conditions was evaluated and presented in TABLE 3. In the second load density study, another GCSF-containing sample (Sample 2) was used. In this second study, the load and column jacket were controlled with a chiller at 10° C. Sample 2 was loaded onto a DEAE column with increasing load densities ranging from 5 g/L to 17 g/L. GCSF yield and product quality were evaluated and presented in TABLE 4. Based upon the data presented in TABLE 3 and TABLE 4, the load density range was established at 8 g/L to 15 g/L.

TABLE 3
Influence of load density on GCSF yield-Sample 1.

| | % Yield |
|---|---|
| 10 g/L load density* | 61.9 |
| 15 g/L load density* | 55.5 |
| 20 g/L load density* | 49.8 |
| 25 g/L load density* | 46.9 |
| Room temp load-11 g/L | 53.6 |
| load density | |
| Cold load (on ice)-11 g/L | 57.2 |
| load density | |

*Performed with chilled load

TABLE 4
Influence of load density on GCSF yield
and product quality - Sample 2.

| | | Reversed Phase Purity | | | IEC | | |
|---|---|---|---|---|---|---|---|
| Load Density | % | % | % | % | % | % | % |
| (g/L) | Yield | LHS | Main | MHS | Acidic | Main | Basic |
| Load | | 8.61 | 73.68 | 17.71 | 6.48 | 93.52 | 0.00 |
| 5 | 35.5 | 2.65 | 93.07 | 4.28 | 5.47 | 94.53 | 0.00 |
| 8 | 83.5 | 2.72 | 93.34 | 3.93 | 5.08 | 94.92 | 0.00 |
| 10 | 62.4 | 2.35 | 93.84 | 3.81 | 4.92 | 95.08 | 0.00 |
| 12 | 67.5 | 2.34 | 94.02 | 3.64 | 5.05 | 94.95 | 0.00 |
| 15 | 72.2 | 2.51 | 93.51 | 3.98 | 5.30 | 94.70 | 0.00 |
| 17 | 69.3 | 2.57 | 93.49 | 3.94 | 5.12 | 94.88 | 0.00 |

Note:
LHS: more hydrophobic species,
MHS: less hydrophobic species

### DEAE Fractionation

Upon completion of loading the samples on the AEX chromatographic material and elution of the product from the chromatographic material into a pool fraction, the amount of contaminant (e.g., host cell protein, HCP) in the pool fraction can provide useful information regarding the separation of the product from the contaminants for a given elution condition, such as, pH or conductivity.

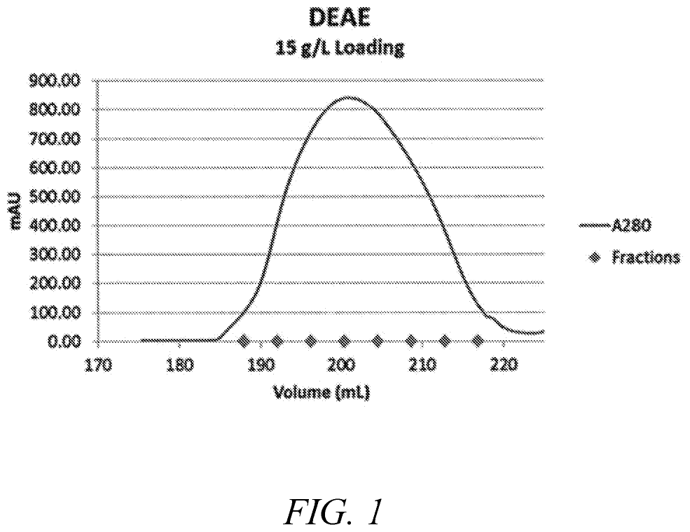

In the experiments presented in TABLE 5, fractionation across the eluted product peak was performed in effort to understand the distribution of product quality across the peak. Generally, GCSF feedstock was loaded on a naive DEAE column at a load density of 15 g/L, and collection of fractions of 0.5 CV in volume from the DEAE resin was initiated at 0.5 OD at 280 nm and stopped when the OD dropped to 0.5. These fractions were subsequently analyzed by reversed phase (RP) and for host cell protein content. A summary of the results are presented in FIG. 1 and TABLE 5. It was observed that (1) the percentage of more hydrophobic species (MHS) was increased in the fractions across the peak; (2) host cell protein (HCP) per protein content was highest at the beginning and end of the pool, indicating that distribution is relatively consistent across the peak.

TABLE 5
Fractionation across DEAE.

| | Reversed Phase Purity | | | HCP |
|---|---|---|---|---|
| Fraction | % LHS | % Main | % MHS | ppm |
| Load | 10.41 | 77.75 | 11.84 | |
| 1 | 9.83 | 80.23 | 9.93 | 104 |
| 2 | 8.41 | 81.65 | 9.94 | 76.5 |
| 3 | 6.86 | 83.96 | 9.18 | 43.1 |
| 4 | 6.06 | 84.60 | 9.34 | 50.7 |
| 5 | 5.69 | 83.60 | 10.72 | 63.4 |
| 6 | 5.57 | 82.74 | 11.69 | 84.9 |
| 7 | 6.53 | 78.00 | 15.46 | 149 |

Note:
LHS: less hydrophobic species,
MHS: more hydrophobic pecies,
HCP: host cell protein.

### DEAE Column Lifetime

DEAE resin lifetime was evaluated as follows. Resin lifetime with the revised temperature conditions of the load and column was evaluated with two different feedstocks, i.e. Engineering 1 material and Engineering 2 material, under similar conditions.

Cycling was evaluated with Engineering 1 material at a load density of 15 g/L with the load and column jacket temperature at a set-point of 10 ° C. Pools from the Engineering 1 cycling studies were analyzed for yield and the result is presented in TABLE 6.

TABLE 6
Engineering 1 DEAE cycling study.

| Cycle | % Yield |
|---|---|
| 1 | 76.2 |
| 2 | 76.3 |
| 3 | 77.7 |
| 4 | 76.7 |
| 5 | 68.0 |
| 6 | 79.2 |
| 7 | 80.0 |

Cycling was also evaluated with Engineering 2 material under similar conditions. Differences between of Engineering 1 and Engineering 2 occurred in the handling of the load material to control feedstock product quality changes over time. Engineering 1 cycling had the first 5 cycles with a load held at 10° C. and a fresh thaw for the remaining 2 cycles. Engineering 2 cycling had a fresh load thawed every 2 cycles. Pools from the Engineering 2 cycling studies were analyzed for yield and product quality (TABLE 7). After the first cycle, there was a slight yield increase proportionally with cycling number. Recommended lifetime based upon these studies is 8 cycles, but there is a possibility of extension. The most noticeable change was the increase in aggregate level with cycling. More cycles would be necessary to determine if this is significant.

TABLE 7
Engineering 2 DEAE cycling study.

| | | Reversed Phase Purity | | | IEC | | | SEC | | |
|---|---|---|---|---|---|---|---|---|---|---|
| | % | % | % | % | % | % | % | % | % | % |
| Cycle | Yield | LHS | Main | MHS | Acidic | Main | Basic | HMW | Main | LMW |
| 1 | 51.4 | 2.51 | 93.56 | 3.93 | 4.65 | 95.35 | 0.00 | 0.26 | 99.74 | 0.00 |
| 2 | 69.4 | 2.52 | 93.45 | 4.03 | 4.67 | 95.33 | 0.00 | 0.28 | 99.72 | 0.00 |
| 3 | 66.7 | 2.48 | 93.46 | 4.05 | 4.57 | 95.43 | 0.00 | 0.28 | 99.72 | 0.00 |
| 4 | 68.6 | 2.52 | 93.42 | 4.06 | 4.73 | 95.27 | 0.00 | 0.32 | 99.68 | 0.00 |
| 5 | 73.8 | 2.49 | 93.40 | 4.11 | 4.67 | 95.33 | 0.00 | 0.28 | 99.72 | 0.00 |
| 6 | 78.4 | 2.57 | 93.19 | 4.25 | 4.85 | 95.15 | 0.00 | 0.32 | 99.68 | 0.00 |
| 7 | 77.5 | 2.56 | 93.18 | 4.26 | 4.90 | 95.10 | 0.00 | 0.34 | 99.66 | 0.00 |
| 8 | 78.8 | 2.69 | 93.02 | 4.29 | 4.88 | 95.12 | 0.00 | 0.36 | 99.64 | 0.00 |

Based upon the data above, an improved DEAE chromatography process for GCSF purification was identified. Details of the improved process are described in TABLE 8 and 9 below. In the improved process, AEX operations can be generally performed using the chromatographic parameters described in Table 8. Buffers solutions and running conditions are also described in Table 9.

Most notable characteristics of the improved process include placing the load and column under a 10° C. jacket set-point. Binding capacity was set at 8 g/L-15 g/L with an 8 cycle lifetime. Without being bound to any particular theory, there is a potential to extend the lifetime of this column as the chilled operation has offered improvement in the ability to regenerate the column.

TABLE 8
Standard anion exchange chromatography parameters.

| Resin | DEAE Sepharose Fast Flow |
|---|---|
| Capacity | ≤15.0 g/L |
| Load | B1HC 2 Pool |
| Load temperature setpoint | 10° C. |
| Column jacket temperature setpoint | 10° C. |
| Elution | Bind and step elute |
| Flow rate | 150 cm/h |
| Pooling | 0.5 OD−0.5 OD |
| Post-pH adjustment | pH 5.0 with 2M Acetic Acid |

TABLE 9
AEX running conditions.

| | | Flow Rate | Duration |
|---|---|---|---|
| Step | Buffer/Solution | (cm/hr) | (CVs) |
| Equilibration | 40 mM Tris pH 7.7 | 150 | 6 |
| Load | B1HC 2 Pool | 150 | As needed to 15 g/L |
| Wash | 40 mM Tris pH 7.7 | 150 | 10 |
| Elution | 40 mM Tris, 50 mM NaCl | 150 | 7 |
| | pH 7.7 | | |
| Strip | 2M NaCl | 50 | 5 |
| Sanitization | 0.5 N Sodium Hydroxide | 50 | 5 |
| Storage | 0.1 N Sodium Hydroxide | 150 | 4 |

### Example 2

### Purification of GCSF Using a Temperature-Controlled AEX Chromatography Procedure

This Example describes the purification of recombinant human GCSF (rhGCSF) using a temperature-controlled AEX chromatography procedure in accordance with some embodiments of the methods disclosed herein. In these experiments, DEAE chromatography was performed in a bind-and-elute mode under cold temperature conditions with a jacketed DEAE Sepharose® Fast Flow (FF). A water-jacketed column was packed with DEAE Sepharose® FF resin and was equilibrated with 40 mM Tris pH 7.6. The column jacket temperature and load vessel jacket temperature were both set to 10±3° C. The DEAE column is operated at a linear flow rate of 150 cm/hr for all phases except the strip and sanitization steps, which are performed at 50 cm/hr.

The column was first equilibrated with an equilibration buffer (40 mM Tris pH 7.6) for 6 column volumes (CVs), then a refolded GCSF sample which had been buffer exchanged into 40 mM Tris pH 7.6, was loaded onto the jacketed column to a maximum protein load density of 15 g of GCSF per liter of resin (15 g/L).

Upon completion of GCSF binding to the DEAE Sepharose resin, the bound GCSF was first washed with 10 CVs of equilibration buffer, and subsequently eluted from the DEAE resin with 10 CVs of an elution buffer (40 mM Tris, 50 mM sodium chloride, pH 7.7). Collection of the GCSF pools eluted from the DEAE resin was initiated at 0.5 OD at 280 nm and stopped when the OD dropped to 0.5. The column was then stripped with 5 CVs of 2 M sodium chloride, sanitized with 5 CVs 0.5 N sodium hydroxide, and stored with 4 CVs of 0.1 N sodium hydroxide.

### Example 3

### Evaluation and Optimization of AEX Chromatography Parameters

This Example describes the results from a number of robustness studies performed to evaluate and optimize various parameters for the preparation of recombinant hGCSF in a temperature-controlled DEAE chromatography process. These parameters include (1) load density, (2) load pH, (3) load conductivity, (4) equilibration pH, (5) equilibration conductivity, (6) elution pH, (7) elution conductivity, (8) load jacket set point temperature, and (9) column jacket set point temperature. In these experiments, DEAE functions as a capture chromatography column with potential capability of reducing oxidative species, DNA, and E. coli host cell protein (ECP). The results from the experiments described below indicate that certain parameters have an influence on product quality or yield. These studies have either confirmed or lead to a revision of parameter classification from the risk assessment as critical process parameters (CPP), key process parameters (KPP), or non-key parameters (NKP).

All robustness studies described in this Example were conducted using the qualified scale-down model of 1.0 cm×10 cm for DEAE chromatography.

### Study Design

A D-optimal augmentation design was used to define the experimental sets for this study. D-optimal design enables non-linear models and an enhanced ability to evaluate factor combinations over traditional factorial designs. The D-optimal design minimizes the covariance of the parameter estimates through an iterative search algorithm using JMP™ Software. This design was further optimized to minimize two-factor interactions to only interactions with potential to significantly affect the process.

This design of experiments approach for the evaluation of 9 parameters resulted in 48 experiments of various parameter combinations. Ranges of the parameters evaluated are summarized in TABLE 10.

TABLE 10
Experimental Design Parameter Ranges.

| Parameter | Robustness Study Range |
|---|---|
| Equilibration pH | 7.3-7.9 |
| Equilibration conductivity (mS/cm) | 2-3 |
| Column jacket temperature set-point (° C.) | 7-13 |
| Protein load pH | 7.4-8.0 |
| Protein load conductivity (mS/cm) | 1.5-2.5 |
| Protein load density (g/L) | 6-18 |
| Load jacket temperature set-point (° C.) | 7-13 |
| Elution pH | 7.4-8.0 |
| Elution conductivity (mS/cm) | 7.4-8.2 |

### Data Analysis

In-process data from the pivotal phase campaign were used to evaluate acceptability of the DEAE robustness data for product related impurities. The mean±3 standard deviations of the main peak was the limit established for each product quality related assay such as, reverse phase (RP) purity, size-exclusion chromatography (SEC), and cation exchange (CEX) (see, for example, TABLE 11). The SEC data exhibited little variability in the dataset, therefore a main peak lower limit of 99.0% was used. This was the same limit used to qualify the DEAE scale down model.

TABLE 11
Pivotal campaign in-process data.

| Sample | RP Purity | SEC | CEX |
|---|---|---|---|
| | (% Main) | (% Main) | (% Main) |
| Sample #1 | 92.25 | 99.7 | 94.84 |
| Sample #2 | 91.40 | 99.8 | 94.23 |
| Sample #3 | 91.50 | 99.7 | 93.84 |
| Sample #5 | 91.87 | 99.7 | 93.35 |
| Mean | 91.75 | 99.72 | 94.06 |
| Standard Deviation | 0.39 | 0.03 | 0.63 |
| Mean ± 3SD | 90.60-92.91 | 99.64 | 92.18-95.95 |

JMP™ Software was used to model the resulting data for this study. A standard least squares approach was used to evaluate the effect of each process input (see, TABLE 10) on a selected process output (RP Purity, SEC, CEX, ECP, DNA, and yield). This analysis generated an effect summary table with parameters ranked in order of decreasing statistical effect upon the output. A p value <0.01 indicates that the process input has a statistically significant influence on the output.

The statistically significant parameters were evaluated in the software's Prediction Profiler. This analysis generated a profile trace which enables visualization of the predicted response of a change in one parameter while the other parameters are held constant. Desirability profiling was enabled in the graphical representation. This analysis allowed for a range setting of the output. The minimum (0) and maximum (1) range settings for RP Purity, SEC, and CEX were defined as mean±3SD as in TABLE 10. The desirability trace is equivalent to the predicted response trace for each parameter if the desired output is the mean of the range.

The statistically significant parameters were also evaluated in the software's Contour Profiler. This analysis generated response contours of two factors at a time, which enables visualization of the predicted responses within a defined output range when two factors have a combined influence.

### Analytical Methods

The following assays were used to evaluate a number of quality attributes of the GCSF samples obtained from the DEAE chromatography purification: (1) product-related impurities assays was performed using C3 Reverse Phase HPLC; (2) determination of purity of GCSF samples was performed using size exclusion chromatography (SEC); (3) determination of charge heterogeneity of GCSF samples was performed using CEX HPLC; (4) measurement of E. coli host cell protein (ECP) in GCSF samples was performed using enzyme-linked immunosorbent assay (ELISA); (5) concentration of residual E. coli host cell DNA was assayed using Q-PCR.

### Observations

JMP® Software (SAS) was used for statistical analysis of the data generated from the DEAE robustness experiments. Outputs analyzed in these experiments include RP Purity % main, SEC % main, CEX % main, ECP, and DNA. Nine parameters were found to have a significant influence (p value <0.01) as follows (in order of decreasing effect): (1) equilibration pH, (2) column temperature set-point, (3) equilibration conductivity, (4) elution conductivity, (5) load conductivity, (6) elution pH, a (7) combined interaction of load density with load pH, (8) a combined interaction of load density with equilibration pH, and (9) a combined interaction of equilibration conductivity with elution conductivity.

These identified factors were further analyzed to determine their impact to the RP Purity range limits of 90.60 to 92.91% (data not shown). It was demonstrated in this analysis that although these nine parameters impact the RP Purity % main, their influence would not result in an RP Purity % main below the lower limit of 90.60%.

The identified factors were also analyzed in the contour profiler to determine the response ends based upon a high/low limit (data not shown). The contour plots in these analyses confirmed that RP Purity % main will be within the defined range for the parameter ranges tested (see, e.g., TABLE 10).

### Effects of Parameters on SEC % Main

Experiments were also performed to identify process inputs that have a statistically significant effect on the SEC product quality (p value <0.01). Four parameters were found to have a significant influence as follows (in order of decreasing effect): elution conductivity, elution pH, load density, and a combined interaction of equilibration conductivity with elution conductivity (data not shown).

These identified parameters were further analyzed in the prediction profiler to determine their impact to the SEC range of 99.0% (data not shown). In this analysis, it was observed that although these four parameters impact the SEC % main, their influence would not result in an SEC % main below 99.0%.

The identified parameters were also analyzed in the contour profiler to determine the response ends based upon a high/low limit (data not shown). The contour plots in these analyses confirmed that SEC % main would be within the defined range for the parameter ranges tested (see, e.g., TABLE 10).

### Effects of Parameters on CEX % Main

Additional experiments were performed to identify process inputs that have a statistically significant effect on the CEX product quality (p value <0.01). Four parameters were found to have a significant influence as follows (in order of decreasing effect): elution conductivity, load density, column temperature set-point, and a combined interaction of column temperature set-point and equilibration conductivity.

These identified parameters were further analyzed in the prediction profiler to determine their impact to the CEX range defined to 95.95% (data not shown). This analysis demonstrates that although these four parameters impact the CEX % main, their influence would not result in a CEX % main outside of the 92.18 to 95.95% range.

The identified parameters were also analyzed in the contour profiler to determine the response ends based upon a high/low limit (data not shown). These contour plots confirm that CEX % main will be within the range defined for the parameter ranges tested (TABLE 10).

### Effects of Parameters on ECP

Further experiments were performed to identify process inputs that have a statistically significant effect on the ECP impurity (p value <0.01). Four parameters were found to have a significant influence as follows (in order of decreasing effect): elution pH, elution conductivity, a combined interaction of load temperature set-point and elution pH, and load temperature set-point.

These identified parameters were further analyzed in the prediction profiler to determine their impact to the ECP (data not shown). A range of 200 to 600 ppm was used for this analysis. This analysis demonstrates that elution pH and elution conductivity have a slight influence on ECP content. Clearance of ECP are also evaluated.

The identified parameters were also analyzed in the contour profiler to determine the response ends based upon a high/low limit (data not shown). The low limit was set at 0 ppm and the high limit was set at 423 ppm for the purpose of contour visualization. The contours indicate the DEAE process would result in pools with minimal variance in ECP for the parameter ranges tested (TABLE 10).

Taken together, process characterization was performed around the DEAE chromatography column to build an understanding of the influence of selected parameters on product quality attributes. Nine parameters were identified for evaluation in this robustness study. Modeling of the resulting data led to the identification of parameters with influence on the product quality attributes of RP Purity % main. SEC % main, CEX % main, ECP, and DNA. A summary table of the influential parameters determined can be found in TABLE 12. All parameters with influence on product quality were modeled against product quality limits established from the pivotal phase production testing (mean±3 SD) for RP Purity, SEC, and CEX % main. This modeling predicted all ranges evaluated for each parameter in this study would result in product quality falling within acceptable limits. ECP evaluation indicated influential parameters would result in minimal variability from the ranges evaluated. It was also observed that parameters evaluated had no influence on DNA content in the DEAE pool. Parameters evaluated also did not influence yield of the chromatography operation.

TABLE 12
Parameters Influence on Product Quality.

| | RP Purity | SEC | CEX | | | |
|---|---|---|---|---|---|---|
| Parameter | % main | % main | % main | ECP | DNA | Yield |
| Equilibration pH | Y, a | N | N | N | N | N |
| Equilibration conductivity (mS/cm) | Y, d | d | f | N | N | N |
| Column jacket temperature | Y | N | Y, e | N | N | N |
| set-point (° C.) | | | | | | |
| Protein load pH | a | N | N | N | N | N |
| Protein load conductivity (mS/cm) | Y | N | N | N | N | N |
| Protein load density (g/L) | b, c | Y | Y | N | N | N |
| Load jacket temperature | N | N | N | Y, g | N | N |
| set-point (° C.) | | | | | | |
| Elution pH | Y | Y | Y | Y, h | N | N |
| Elution conductivity (mS/cm) | Y, e | Y, e | Y | Y | N | N |

a: combined interaction with load density
b: combined interaction with load pH
c: combined interaction with equilibration pH
d: combined interaction with elution conductivity
e: combined interaction with equilibration conductivity
f: combined interaction with column jacket temperature set-point
g: combined interaction with elution pH
h: combined interaction with load jacket temperature set-point

Parameter classifications determined by a GCSF downstream risk assessment are summarized in TABLE 13 with the revised classifications from this robustness study. Four parameters identified as NKPs by the risk assessment remain NKPs through this robustness study. These NKPs are equilibration pH, equilibration conductivity, elution pH, and elution conductivity. Although these parameters were originally assessed as NKPs in the risk assessment, they were selected for evaluation to increase knowledge regarding the operation ranges. All of these parameters were found to have various product quality influences on RP Purity % main, SEC % main, and CEX % main. They are classified as NKPs because these pH and conductivity values are well controlled with buffer preparation. Buffer attributes measured to be outside of the operating ranges will not meet release specifications therefore, will not be used in production.

TABLE 13
Parameters Classification and Ranges.

| | Type | | | Accept- |
|---|---|---|---|---|
| | Risk | | Operating | able |
| Parameter | Assessment | Robustness | Range | Range |
| Equilibration pH | NKP | NKP | 7.6 | N/A |
| | | | (7.4-7.8) | |
| Equilibration | NKP | NKP | 2.5 | N/A |
| conductivity (mS/cm) | | | (2.0-3.0) | |
| Column jacket | KPP | KPP | 10 | 7-13 |
| temperature | | | | |
| set-point (° C.) | | | | |
| Protein load pH | NKP | NKP | 7.6 | N/A |
| | | | (7.4-7.8) | |
| Protein load conductivity | NKP | KPP | <3 | <3 |
| (mS/cm) | | | | |
| Protein load density | NKP | KPP | 8-15 | 6-18 |
| (g/L) | | | | |
| Load jacket temperature | KPP | KPP | 10 | 7-13 |
| set-point (° C.) | | | | |
| Elution pH | NKP | NKP | 7.7 | N/A |
| | | | (7.5-79) | |
| Elution conductivity | NKP | NKP | 7.8 | N/A |
| (mS/cm) | | | (7.4-8.2) | |

Both of the jacket temperature set-points for the column and the load were classified as KPPs in the risk assessment. The column jacket temperature has an influence on the RP Purity % main and the CEX % main. The load jacket temperature demonstrated an influence on ECP. The temperature set-point could be suitably controlled with the chiller interface. For this reason, both set points remained classified as KPPs with this study. For both parameters, the operating range is a fixed temperature setting on the chiller. The acceptable range was defined as the ranges evaluated in this robustness study as the ranges tested did not result in product quality outside of acceptability.

The protein load pH remained as a NKP with this robustness study. This parameter only has a combined influence with load density on RP Purity % main so it remains as a NKP as the purity will remain within range. The pH of the DEAE load was controlled through the diafiltration buffer pH used in the UF/DF I operation. There is a 10 diavolume exchange with this buffer.

The protein load conductivity parameter classification was revised through this robustness study. This parameter was assessed as a NKP by the risk assessment. The protein load conductivity has been re-classified as a KPP. Although there is some level of control of this conductivity with the diafiltration buffer, there is also a depth filtration operation prior to the DEAE load which results in a decrease of the load conductivity through filter holdup from pre-use flush with water for injection (WFI) and post-use flush with WFI to recover product. The protein load conductivity influences the RP Purity % main. The operating range and acceptable range are defined as <3 mS/cm. This study evaluated a 1.5-2.5 range. The experimental data described in this Example has confirmed that the RP Purity % main could be well within the product quality range with the conductivity range evaluated for this study. For this reason, the conductivity operation and acceptable range were converted to a rounded <3 mS/cm.

The protein load density has a combined influence on RP Purity % main with load pH and equilibration pH. This parameter also independently influences SEC % main and CEX % main. This parameter was originally identified as a NKP by the risk assessment, but has been reclassified as a KPP based upon its influence to product quality. The acceptable ranges for this parameter have been defined as the range of load densities evaluated in this study. The operating range offers a safety factor on the acceptable range.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for purifying granulocyte colony-stimulating factor (GCSF), the method comprising:
loading a GCSF-containing sample onto a chromatography vessel comprising an anion exchange chromatography (AEX) material capable of binding the GCSF in the sample, wherein the chromatography vessel is placed in a temperature-controlled enclosure and at least a portion of the enclosure of the chromatography vessel is set at a temperature of about 7° C. to about 13° C.; and
eluting the GCSF from the AEX material with an elution buffer to obtain the purified GCSF.

2. The method of claim 1, wherein the GCSF-containing sample is loaded onto the chromatography vessel using a fluidic channel placed in a temperature-controlled enclosure, wherein at least a portion of the enclosure of the fluidic channel is set at a temperature about 7° C. to about 13° C.

3. The method of any one of claims 1 to 2, where the set temperatures of the enclosure of the vessel and the enclosure of the fluidic channel are the same.

4. The method of any one of claims 1 to 2, where the set temperatures of the enclosure of the chromatography vessel and the enclosure of the fluidic channel jacket are different.

5. The method of any one of claims 1 to 4, where at least one of the enclosure of the chromatography vessel and the enclosure of the fluidic channel is set at a temperature of about 10° C.

6. The method of any one of claims 1 to 2, wherein the chromatography vessel is selected from the group consisting of a column, a tank, a packed bed, a fluidized bed, a cartridge an encapsulated membrane, a reservoir, a chamber, a container, and a mixing vessel.

7. The method of any one of claims 1 to 6, wherein the fluidic channel is a tube, a pipe, a bag, a container, a storage tank, a mixing vessel, or other fluid conduction means.

8. The method of any one of claims 1 to 7, wherein the method further comprising, prior to loading of the GCSF sample, equilibrating the AEX material with an equilibration buffer comprising from about 30 mM to about 50 mM Tris, and at pH of about 7.0 to about 8.0.

9. The method of claim 8, wherein the equilibration buffer comprises about 40 mM Tris and at pH of about 7.6.

10. The method of any one of claims 1 to 9, wherein the method further comprising, prior to elution of the GCSF, washing the AEX material with a wash buffer to remove unbound or weakly bound contaminants

11. The method of claim 10, wherein the wash buffer and the equilibration buffer have the same buffer composition.

12. The method of any one of claims 1 to 11, wherein the GCSF-containing sample comprises a loading buffer.

13. The method of claim 12, wherein the loading buffer has a pH of about 7.4 to about 8.0.

14. The method of any one of claims 1 to 13, wherein the loading of the GCSF sample onto the chromatography vessel is carried out at a conductivity ranging between about 1.5 to about 3.0 mS/cm.

15. The method of any one of claims 1 to 14, wherein the elution buffer comprises about 30 mM-60 mM Tris, about 30 mM-80 mM sodium chloride, and a pH of about 7.4 to about 8.0.

16. The method of claim 15, wherein the elution buffer comprises about 40 mM Tris, about 50 mM sodium chloride, and pH of about 7.7.

17. The method of any one of claims 1 to 16, wherein the elution of the GCSF from the AEX material is carried out at a conductivity ranging between about 7.4 to about 8.2 mS/cm.

18. The method of any one of claims 1 to 17, wherein the AEX material comprises diethylaminoethyl (DEAE) ion-exchange chromatography.

19. The method of any one of claims 1 to 18, wherein the AEX material comprises DEAE Sepharose® resin.

20. The method of claim 19, wherein the DEAE Sepharose® resin comprises DEAE Sepharose® Fast Flow resin.

21. The method of any one of claims 1 to 20, further comprising one or more phases of stripping and/or sanitation of the AEX material.

22. The method of any one of claims 18 to 21, wherein the DEAE chromatography is operated at a linear flow rate for all phases.

23. The method of claim 22, wherein the linear flow rate is about 150 cm/hr.

24. The method of any one of claims 21 to 23, wherein at least one of the stripping and sanitization phases is performed at 50 cm/hr.

25. The method of any one of claims 1 to 24, wherein the GCSF is a recombinant human GCSF (hGCSF) or a variant thereof.

26. The method of any one of claims 1 to 25, wherein the sample comprises GCSF obtained from a recombinant eukaryotic cell or a recombinant prokaryotic cell.

27. The method of any one of claims 1 to 26, wherein further comprising at least one additional purification process.

28. The method of claim 27, the at least one additional purification process is selected from the group consisting of affinity chromatography, cation exchange chromatography (CEX), hydroxyapatite chromatography, size exclusion chromatography (SEC), hydrophobic interaction chromatography (HIC), metal affinity chromatography, mixed mode chromatography (MMC), centrifugation, diafiltration, and ultrafiltration.

29. The method of any one of claims 27 to 28, wherein the at least one additional purification process is performed prior to the AEX chromatography process.

30. The method of any one of claims 27 to 28, wherein the at least one additional purification process is performed after the AEX chromatography process.

31. A system for manufacturing GCSF comprising a chromatography vessel comprising an anion exchange chromatography (AEX) material capable of binding GCSF, wherein the chromatography vessel is encased in a temperature-controlled enclosure and at least a portion of the enclosure of the chromatography vessel is set a temperature of about 7° C. to about 13° C.

32. The system of claim 31, further comprising a fluidic channel placed in a temperature-controlled enclosure, wherein at least a portion of enclosure of the fluidic channel is set at a temperature about 7° C. to about 13° C.

33. A granulocyte colony-stimulating factor (GCSF) purified by a method according to any one of claims 1 to 30, or by a system according to any one of claims 31 to 32.

34. A pharmaceutical composition comprising the GCSF of claim 33.

35. The pharmaceutical composition of claim 34, wherein the pharmaceutical compositions is an aqueous composition, a lyophilisate, or a powder.

36. A method for treating or preventing a disease or health condition in a subject in need thereof, the method comprising administering to the subject a GCSF of claim 33 and/or a pharmaceutical composition of claims 34 to 35.

37. The method of claim 36, wherein the disease or health condition is neutropenia.
